(19) United States
(12) Patent Application Publication
(10) Pub. No.: US 2026/0124010 A1
(43) Pub. Date: May 7, 2026

(54) SYSTEMS AND METHODS FOR CONTROL OF A SURGICAL SYSTEM

(71) Applicant: Intuitive Surgical Operations, Inc., Sunnyvale, CA (US)

(72) Inventors: Ashwinram SURESH, San Jose, CA (US); Lawton N. VERNER, Saratoga, CA (US)

(73) Assignee: Intuitive Surgical Operations, Inc., Sunnyvale, CA (US)

(21) Appl. No.: 18/704,369

(22) PCT Filed: Oct. 27, 2022
(86) PCT No.: PCT/US2022/048012

§ 371 Date: Apr. 24, 2024

Related U.S. Application Data
(60) Provisional application No. 63/273,667, filed on Oct. 29, 2021.

(51) Int. Cl. A61B 34/35; A61B 90/00

(52) U.S. Cl. CPC A61B 34/35; A61B 90/06; A61B 2090/062; A61B 2090/065

(57) ABSTRACT

Systems and methods are provided for control of a surgical system. Accordingly, a current operating condition of the instrument with reference to a defined restricted operating condition of the infant instrument is detected. A force feedback coefficient is determined based on the current operating condition of the instrument. A restricted haptic feedback is determined based on the force feedback coefficient and a nominal haptic feedback. On a first event in which the current operating condition of the instrument changes from being outside the restricted operating condition to being inside the restricted operating condition, the operator of the surgical system is provided an indication that restricted haptic feedback is provided to or is available to be provided to the input device.

### CROSS-REFERENCE TO RELATED APPLICATIONS

This application claims priority to and the benefit of U.S. Provisional Application No. 63/273,667, entitled “Systems and Methods for Control of a Surgical System” filed Oct. 29, 2021, which is incorporated herein by reference in its entirety.

### BACKGROUND

The embodiments described herein relate to surgical systems, and more specifically to teleoperated surgical systems. More particularly, the embodiments described herein relate to systems and methods for controlling surgical systems that include a force feedback that may be provided to a system operator.

Known techniques for Minimally Invasive Surgery (MIS) employ instruments to manipulate tissue that can be either manually controlled or controlled via hand-held or mechanically grounded teleoperated medical systems that operate with at least partial computer-assistance (“telesurgical systems”). Many known MIS instruments include a therapeutic or diagnostic end effector (e.g., forceps, a cutting tool, or a cauterizing tool) mounted on an optional wrist mechanism at the distal end of a shaft. During an MIS procedure, the end effector, wrist mechanism, and the distal end of the shaft are typically inserted into a small incision or a natural orifice of a patient to position the end effector at a work site within the patient's body. The optional wrist mechanism can be used to change the end effector's position and orientation with reference to the shaft to perform a desired procedure at the work site. In known instruments, motion of the instrument as a whole provides mechanical degrees of freedom (DOFs) for movement of the end effector and the wrist mechanisms generally provide the desired DOFs for movement of the end effector with reference to the shaft of the instrument. For example, for forceps or other grasping tools, known wrist mechanisms are able to change the pitch and yaw of the end effector with reference to the shaft. A wrist may optionally provide a roll DOF for the end effector, or the roll DOF may be implemented by rolling the shaft. An end effector may optionally have additional mechanical DOFs, such as grip or knife blade motion. In some instances, wrist and end effector mechanical DOFs may be combined. For example, U.S. Pat. No. 5,792,135 (filed May 16, 1997) discloses a mechanism in which wrist and end effector grip DOFs are combined.

Force sensing surgical instruments are known and together with associated telesurgical systems may deliver haptic feedback during a MIS procedure to a surgeon performing the procedure. The haptic feedback may increase the immersion, realism, and intuitiveness of the procedure. For effective haptics rendering and accuracy, force sensors may be placed on a medical instrument and as close to the anatomical tissue interaction as possible. One approach is to include a force sensor unit having electrical strain sensors (e.g., strain gauges) at a distal end of a medical instrument shaft to measure strain imparted to the medical instrument. The measured strain can be used to determine the force imparted to the medical instrument and as input upon which the desired haptic feedback may be generated.

FIG. 1A shows one example of a known force sensor unit that includes a cantilever beam 810 attached between the instrument distal tip component 510 (e.g., in some cases a clevis or other wrist or end effector component) and the instrument shaft 410 that extends back to the mechanical structure. As illustrated, strain sensors 830 are coupled to the beam to measure strain in X- and Y-directions (arbitrary Cartesian directions that are orthogonal to each other and to a longitudinal axis of the beam and instrument shaft). For example, the strain sensors can include full-Wheatstone bridges (full-bridges). In some cases, the strain sensors are split into two sets, one on the distal end of the beam and the other on the proximal end of the beam in order to reject common-modes. Because the beam is secured to a distal portion of the instrument shaft, the strain sensors sense strain on the beam orthogonal to a longitudinal axis of the shaft. A force F (FIG. 1B) applied orthogonal to the beam (i.e., an X or Y force) is determined by subtracting strain measurements determined by the full-bridges at the proximal and distal end portions of that side face of the beam.

During the employment of the medical instrument however, certain operating conditions may be encountered under which the output of the force sensor unit may not accurately reflect the force imparted to the medical instrument. The operating conditions may, for example, correspond to the positioning of the medical instrument, an operation being performed by the medical instrument, and/or a fault condition. The inaccuracies that may be encountered may limit the ability of the telesurgical system to deliver accurate haptic feedback to the surgeon performing the procedure.

For example, in certain positions, the strain indicated by the strain sensors may be less than the expected strain that will be imparted to the medical instrument in response to the force F applied to the distal tip component 510. More specifically, some known force sensing medical instruments may include or be used with a substantially stiff structure 901 that at least partially circumscribes the beam 810. For example, some known force sensing medical instruments may include a protective shroud that covers the strain sensors 830 and their associated wires during use. Additionally, some known force sensing instruments may include a cannula for facilitating delivery of the force sensing instrument to the surgical site. The structure 901 may, in other words, be a structure that does not deflect to the same degree as the beam 810. To ensure the beam 810 remains cantilevered for accurate force sensing, the structure 901 (e.g., the protective shroud and/or the cannula) may not be directly coupled to the distal tip component 510. Instead, the structure 901 may be separate from the distal tip component to allow the beam to deflect when the force F is applied (see FIG. 1B). In certain situations, however, the distal end of the structure 901 may contact the beam (or a portion of the medical instrument circumscribing the beam) or the distal tip component, thereby limiting bending of the beam. FIG. 1B shows one example, in which the beam 810 is displaced in the X direction such that it contacts one side of the distal end of the structure 901 (e.g., the shroud), which limits or prevents further bending of the beam 810 in the X direction by an amount that is dependent upon, for example, how rigid the structure 901 is and the relative stiffness between the structure 901 and the beam 810.

Although limiting the displacement of the beam can advantageously prevent overload of the beam 810 and/or the strain sensors 830, we have discovered that such known systems that engage the beam at a single point can cause a change in the strain distribution over the length of the beam 810. In other words, the beam 810 no longer functions as a cantilevered beam. As a result, the strain sensors 830 produce signals that do not accurately represent the force F applied to the distal tip component. Specifically, we have discovered that in certain situations the contact between the distal end of the structure 901 (e.g., the protective shroud and/or the cannula) and the beam 810 (or the distal tip component 510) may cause distortion of the signals produced by the strain sensors 830. In certain situations, the distortion can cause the force sensed by the strain sensors 830 to be in the opposite direction of the force F actually applied to the distal tip component 510 (this phenomenon can be referred to as “force inversion”).

FIGS. 2A and 2B show free body diagrams of example known force sensing medical instrument of FIGS. 1A and 1B to further illustrate this example of force distortion. As shown in FIG. 2A, the contact between the shroud and the beam can be modeled as a single point contact (at GND 2). In FIG. 2A, the distance L represents the distance from the base of the beam 810 (point GND 1) to the point where the shroud 901 contacts the beam 810 (point GND 2). The distance D represents the distance between the point where the shroud 901 contacts the beam 810 (point GND 2) and where the force F is applied to or by the distal tip component 510. FIG. 2B is a free-body diagram of the beam showing exaggerated deflection of the beam in such a condition as a result of the contact at point GND 2. As shown, we have discovered that the strain distribution along the top surface of the beam transitions from a region of compression to a region of tension, which causes the signals from the strain sensors 830 to inaccurately represent the applied force F. FIG. 2C shows the modeled forces with the beam “cut” at point GND 2 for purposes of analyzing the force and bending moment of the beam. FIG. 2C shows the reactive force FR produced by the single point contact, the effective force FE, and the effective moment ME produced by the cantilever coupling to the shaft. By modeling the beam at the point of contact (at GND 2), the additional deflection (i.e., beyond this point of contact) can be considered as zero. Using the static and deflection equations shows that there are two different strain profiles over the entire beam length. The strain profile (ε) on the top side of the beam for the beam length l being between ( ) and L is given by Eq. (1), where E is the modulus of elasticity of the beam and I is the moment of inertia of the XY cross-section of the beam:

$$ε ( l ) = - \frac{{F}_{E} ( L - l )}{EI} + \frac{{M}_{E}}{EI} \qquad Eq . ( 1 )$$

The strain profile (ε) on the top side of the beam for the beam length l being between L and L+d is given by Eq. (2):

$$ε ( l ) = - \frac{F ( L + d - l )}{EI} \qquad Eq . ( 2 )$$

Thus, at certain locations along the beam 810, the strain sensors 830 produce a signal associated with FE and not the actual force F. Because FE is acting in the opposite direction of the actual force F, which is due to the FR being larger than F, the result is a distortion (and even an inversion of force direction) of the measured force. FIG. 3A is a graph showing the strain along the top of the beam 810 along the length of the beam based on Eq. (1) and Eq. (2) for the condition when the beam 810 contacts the structure 901 at the single point of contact (GND 2). To further illustrate force distortion, FIG. 3B is a graph showing measured force (based on the strain signals) as a function of the actual force applied. As shown, when the beam 810 is not in contact with the shroud and/or the cannula (e.g., the substantially stiff structure 901), for example, when the actual force applied does not cause sufficient bending of the beam 810 to result in the displacement of the beam 810 being affected by the cannula 901, the relationship between the measured force and the actual force is linear, which allows for an accurate calibration (i.e., based on the slope of the line). At conditions where the beam 810 is in contact with the shroud/cannula (as shown in FIG. 1B), however, the measured force decreases as the actual force increases.

When the measured force is used to produce haptic feedback to a person operating an instrument that includes the beam (e.g., at a master controller), this measured force distortion/force inversion problem can result in an undesirable positive feedback loop, which could cause unexpected or undesirable movement at the master controller. This discovery is more fully described in in U.S. Patent Application No. U.S. 63/026,320 (filed May 18, 2020), entitled “Hard Stop that Produces a Reactive Moment Upon Engagement for Cantilever-Based Force Sensing,” which is incorporated herein by reference in its entirety for all purposes.

In view of the aforementioned, the art is continuously seeking new and improved systems and methods for control of a surgical system.

### SUMMARY

This summary introduces certain aspects of the embodiments described herein to provide a basic understanding. This summary is not an extensive overview of the inventive subject matter, and it is not intended to identify key or critical elements or to delineate the scope of the inventive subject matter.

In one aspect, the present disclosure is directed to a method of control for a surgical system. The surgical system may include a controller, an input device, and a medical instrument operably coupled to be controlled by the input device via the controller. The method may include, detecting, via the controller, a current operating condition of the instrument with reference to a defined restricted operating condition of the instrument. The controller may also determine a force feedback coefficient based on the current operating condition of the instrument. Additionally, the controller may determine a restricted haptic feedback based on a nominal haptic feedback and on the force feedback coefficient. During a first condition in which the current operating condition of the instrument is outside the restricted operating condition of the instrument, the controller may provide the nominal haptic feedback to the input device. Additionally, on a first event in which the current operating condition of the instrument changes from being outside the restricted operating condition of the instrument to being inside the restricted operating condition of the instrument the controller may provide to an operator of the surgical system an indication that restricted haptic feedback is provided to, or is available to be provided to, the input device.

In an embodiment, during a second condition in which the current operating condition of the instrument is inside the restricted operating condition of the instrument, the method may include providing, via the controller, the restricted haptic feedback to the input device.

In an additional embodiment, during a second condition in which the current operating condition is inside the restricted operating condition, and on a second event in which an input by the operator acknowledging the indication of the restricted feedback is received at the controller, the method may include providing, via the controller, the restricted haptic feedback to the input device.

In a further embodiment, the restricted operating condition may be a portion of an operating range of the instrument in which a determination by the controller of a force acting on the instrument deviates from the actual force acting on the instrument. As such, in an embodiment, detecting the current operating condition of the instrument includes determining, via the controller, a distance between a portion of the instrument and a defined reference location on a cannula of the surgical system.

In yet another embodiment, determining the force feedback coefficient may include defining, via the controller, the force feedback coefficient based on a gain function. Additionally, in an embodiment, the controller may determine the gain function based on at least one of an acceleration of a portion of the instrument, a power generated by the surgical system, or a direction of a change in force.

In an embodiment, providing the indication that restricted haptic feedback is provided or available may include generating, via the controller, a graphical indication of a deviation of the restricted haptic feedback from the nominal haptic feedback. The graphical indication may be maintained within a field of view of the operator when the instrument is inside the restricted operating condition.

In an additional aspect, the present disclosure is directed to a method of control for a surgical system. The surgical system may include a controller, an input device, and a medical instrument operably coupled to be controlled by the input device via the controller. The method may include, detecting, via the controller, a current operating condition of the instrument with reference to a defined restricted operating condition of the instrument. During a first condition in which the current operating condition of the instrument is outside the restricted operating condition of the instrument, the controller may provide a nominal haptic feedback to the input device. The method may also include initiating a first event transition wherein the current operating condition of the instrument changes from being outside the restricted operating condition of the instrument to being inside the restricted operating condition of the instrument. The controller may determine: a rate of adjustment of the nominal haptic feedback associated with the first event transition. During a second condition in which the current operating condition of the instrument is inside the restricted operating condition of the instrument, the controller may provide a restricted haptic feedback to the input device based on the rate of adjustment and the nominal haptic feedback. Additionally, the controller may provide an operator of the surgical system an indication that restricted haptic feedback is provided.

In an embodiment, the restricted operating condition may be associated with an activation of an energized medical instrument of the surgical system. Accordingly, the first event transition may be initiated upon receipt of a command signal from the operator of the surgical system initiating an operation that utilizes the energized medical instrument.

In an additional embodiment, providing the restricted haptic feedback may include establishing the haptic feedback in accordance with a feedback-restriction interval. The feedback-restriction interval may be based on a nominal duration of the activation of the energized medical instrument.

In a further embodiment, determining the rate of adjustment of the nominal haptic feedback may include defining, via the controller, the rate of adjustment based on a gain function. In an embodiment, the controller may determine the gain function based on at least one of an acceleration of a portion of the instrument, a power generated by the surgical system, or a direction of a change in force.

In yet another embodiment, the method may include initiating a second event transition wherein the current operating condition of the instrument changes from the second condition to the first condition. In such an embodiment, the controller may determine a rate of adjustment of the restricted haptic feedback associated with the second event transition. Additionally, the controller may transition the restricted haptic feedback to the nominal haptic feedback in accordance with the rate of adjustment.

In an additional aspect, the present disclosure is directed to a method of control for a surgical system. The surgical system may include a controller, an input device, and a medical instrument operably coupled to be controlled by the input device via the controller. The method may include, detecting, via the controller, a current operating condition of the instrument with reference to a defined restricted operating condition of the instrument. During a first condition in which the current operating condition of the instrument is outside the restricted operating condition of the instrument, the controller may provide a nominal haptic feedback to the input device. On a first event in which the current operating condition of the instrument changes from being outside the restricted operating condition of the instrument to being inside the restricted operating condition of the instrument, the controller may pause at least one operation of the surgical system. On the first event, the controller may provide to an operator of the surgical system an indication that restricted haptic feedback is available to be provided to the input device. The controller may receive a first confirmation input by the operator acknowledging the indication of the restricted haptic feedback availability. During a second condition in which the current operating condition is inside the restricted operating condition and upon receipt of the confirmation input the controller may provide the restricted haptic feedback to the input device. Additionally, during the second condition and upon receipt of the confirmation input, the at least one operation of the surgical system may be resumed.

In an embodiment, on a second event in which the current operating condition of the instrument changes from being inside the restricted operating condition of the instrument to being outside the restricted operating condition of the instrument, the controller may pause the operation(s) of the surgical system. On the second event, the controller may provide to the operator of the surgical system an indication that the nominal haptic feedback is available to be provided to the input device. The controller may receive a second confirmation input by the operator acknowledging the indication of the nominal haptic feedback availability. During the first condition and upon receipt of the second confirmation input, the controller may provide the nominal haptic feedback to the input device. Additionally, during the first condition and upon receipt of the second confirmation input, resuming, via the controller, the at least one operation of the surgical system.

In an additional embodiment, the restricted operating condition may correspond to a fault condition. In such an embodiment, the method may include receiving, via the controller, a communication signal associated with the fault condition, the fault condition being at least one of a sensor fault, a communications fault, or a haptic system fault.

In a further embodiment, the confirmation input may include at least one of an operator gesture, an engagement between an operator's head and a user interface of the surgical system, a touchpad input, a button activation, a pedal activation, a button and pedal activation combination, or a crossing of a haptic barrier.

In yet another embodiment, providing the restricted haptic feedback may include limiting the restricted haptic feedback to a percentage of a nominal haptic feedback level, the percentage being less than 100 percent. Such an embodiment may include dynamically reducing the percentage of the nominal haptic feedback level based on a gain function.

In additional aspects, the present disclosure is directed to multiple embodiments of a surgical system. The surgical system includes a controller. The controller is operably coupled to an input device. The surgical system also includes a manipulator unit operably coupled to the input device via the controller. A medical instrument is supported by the manipulator unit and is also operably coupled to the controller. The controller includes at least one processor and a haptic feedback module configured to perform a plurality of operations. The plurality of operations may include any of the methods, procedures, and/or operations described herein.

These and other features, aspects and advantages of the present invention will become better understood with reference to the following description and appended claims. The accompanying drawings, which are incorporated in and constitute a part of this specification, illustrate embodiments of the invention and, together with the description, serve to explain the principles of the invention.

### BRIEF DESCRIPTION OF THE DRAWINGS

FIGS. 1A and 1B are diagrammatic illustration of a portion of a known medical device including a force sensor unit in a first configuration (FIG. 1A) and a second configuration (FIG. 1B).

FIGS. 2A and 2B are free-body diagrams of the portion of the medical device shown in FIGS. 1A and 1B in the first configuration (FIG. 2A) and showing an exaggerated bending (FIG. 2B).

FIG. 2C is a free-body diagram of the portion of the medical device shown in FIGS. 1A and 1B being analyzed at a point of contact.

FIG. 3A is a graph showing the surface strain along the length of a beam of a force sensor unit when a single point of contact occurs.

FIG. 3B is a graph showing the measured force (Y-axis) as a function of the actual force (X-axis) to demonstrate force distortion.

FIG. 4 is a plan view of a minimally invasive teleoperated medical system according to an embodiment being used to perform a medical procedure such as surgery.

FIG. 5 is a plan view of the minimally invasive teleoperated medical system of FIG. 4 being used to perform a medical procedure such as surgery.

FIG. 6 is a perspective view of a user control console of the minimally invasive teleoperated surgery system shown in FIG. 5 according to an embodiment.

FIG. 7 is a perspective view of an input control device of the user console shown in FIG. 6 according to an embodiment.

FIG. 8 illustrates a displayed view of a surgical site as presented to an operator of the minimally invasive teleoperated surgery system by the user control console shown in FIG. 6 according to an embodiment.

FIG. 9 is a perspective view of an optional auxiliary unit of the minimally invasive teleoperated surgery system shown in FIG. 5.

FIG. 10 is a front view of a manipulator unit, including a plurality of instruments, of the minimally invasive teleoperated surgery system shown in FIG. 5.

FIG. 11 is a diagrammatic illustration of a portion of a medical including a force sensor unit.

FIG. 12 is a perspective view of a medical device according to an embodiment.

FIG. 13 is a side view of a portion of the medical device of FIG. 12 according to an embodiment.

FIG. 14 is a cross-sectional view of a cannula for use with the medical device of FIG. 12 according to an embodiment.

FIG. 15 is a flow chart of a method of control for a surgical system according to an embodiment.

FIG. 16 is a diagrammatic illustration of an operating range of a portion of a medical device of a minimally invasive teleoperated surgery system according to an embodiment.

FIG. 17 is a graph depicting a modification of a haptic feedback level relative to a position of a portion of a medical device according to an embodiment.

FIG. 18 is a graph depicting a modification of a haptic feedback level relative to a position of a portion of a medical device according to an embodiment.

FIG. 19 is a flow chart of a method of control for a surgical system according to an embodiment.

FIG. 20 is a graph depicting a modification of a haptic feedback level relative to time according to an embodiment.

FIG. 21 is a flow chart of a method of control for a surgical system according to an embodiment.

FIG. 22 is a schematic illustration of a controller for use with a minimally invasive teleoperated surgery system according to an embodiment.

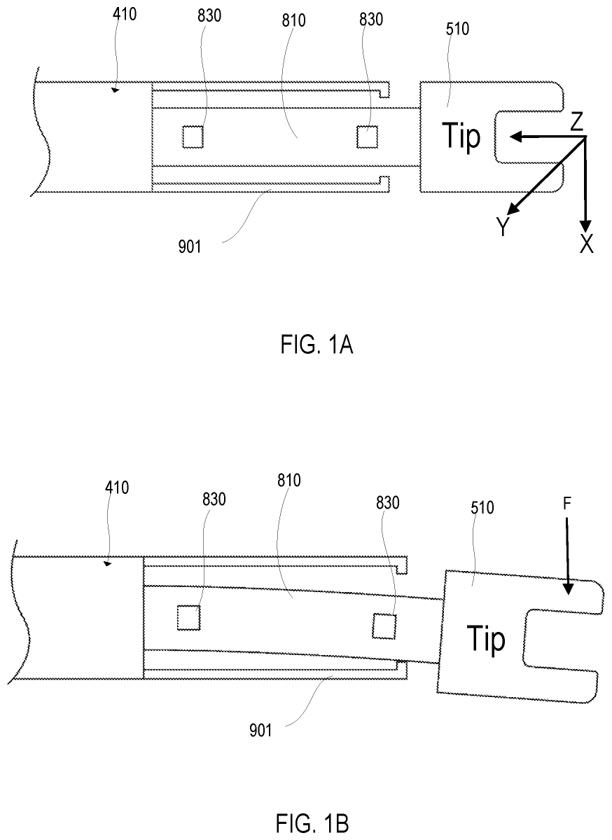

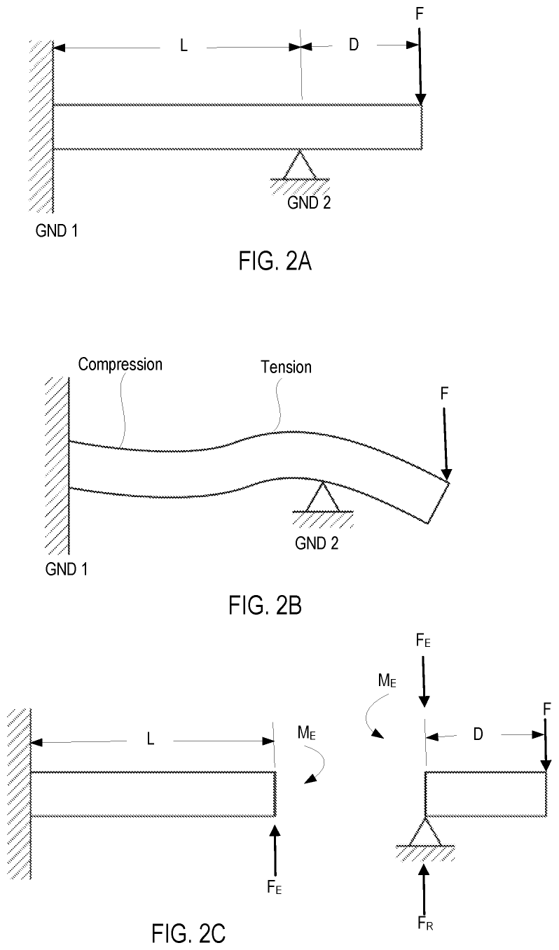

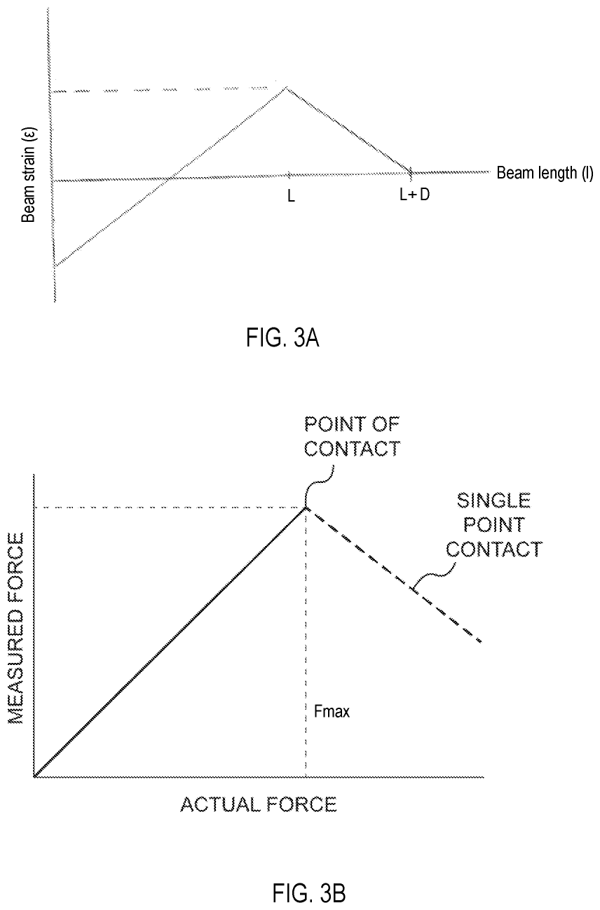

### DETAILED DESCRIPTION

Reference now will be made in detail to embodiments of the invention, one or more examples of which are illustrated in the drawings. Each example is provided by way of explanation of the invention, not limitation of the invention. In fact, it will be apparent to those skilled in the art that various modifications and variations can be made in the present invention without departing from the scope or spirit of the invention. For instance, features illustrated or described as part of one embodiment can be used with another embodiment to yield a still further embodiment. Thus, it is intended that the present invention covers such modifications and variations as come within the scope of the appended claims and their equivalents.

The embodiments described herein can advantageously be used in a wide variety of grasping, cutting, and manipulating operations associated with minimally invasive surgery. The medical instruments or devices of the present application enable motion in three or more degrees of freedom (DOFs). For example, in some embodiments, an end effector of the medical instrument can move with reference to the main body of the instrument in three mechanical DOFs, e.g., pitch, yaw; and roll (shaft roll). There may also be one or more mechanical DOFs in the end effector itself, e.g., two jaws, each rotating with reference to a clevis (2 DOFs) and a distal clevis that may rotate with reference to a proximal clevis (one DOF). Thus, in some embodiments, the medical instruments or devices of the present application may enable motion in six DOFs. The embodiments described herein further may be used to deliver a modified force feedback to a system operator in response to forces exerted on (or by) a distal end portion of the instrument during use under certain operating conditions.

Generally, the present disclosure is directed to systems and methods for controlling a surgical system such as a minimally invasive teleoperated surgery system. In particular, the present disclosure may include a system and methods that may facilitate the modification of the haptic feedback delivered to the operator of the surgical system in relation to a restricted feedback condition of the surgical system. The restricted feedback condition may correspond to a condition of the surgical system wherein the haptic feedback generated based on the measured force may not accurately reflect the forces acting on the instrument. For example, the restricted feedback condition may correspond to a portion of an operating range of the medical device in which the force measured by the surgical system deviates from the actual force exerted on (or by) the distal end of the medical device. The restricted feedback condition may also correspond to a particular operation of the medical device, such as an energizing of the medical device (e.g., during certain cutting and/or cauterization procedures). By way of an additional example, the restricted feedback condition may correspond to a fault condition, such as a sensor fault, a communications fault, and/or a haptic system fault.

As disclosed herein, when the position, condition, and/or operation of the medical device of the surgical system is in the restricted feedback condition, the force feedback (e.g., haptic feedback) delivered to the operator of the surgical system may be reduced/limited relative to a nominal haptic feedback. The reduction/limiting (e.g., disabling) of the haptic feedback may facilitate continued, accurate control of the surgical system by the operator under conditions wherein the haptic feedback may otherwise be inaccurate and/or unreliable.

In addition to affecting the haptic feedback provided to the operator of the surgical system, the systems and methods disclosed herein may also pause (e.g., freeze or hold in place) an operation of the surgical system when at or approaching a transition between a restricted feedback condition and an unrestricted feedback condition. An indication of the transition may be presented to the operator. Upon acknowledgment of the operator, the operation of the surgical system may be resumed and the appropriate haptic feedback may be provided to the operator. For example, when transitioning from the unrestricted feedback condition to the restricted feedback condition, upon acknowledgment, the haptic feedback delivered to the operator may be reduced (e.g., disabled). Similarly, when transitioning from the restricted feedback condition to the unrestricted feedback condition, upon acknowledgment, nominal haptic feedback may be delivered to the operator. It should be appreciated that the pausing of the operation of the surgical system until the acknowledgment of the modification of the haptic feedback is received may facilitate transitions between feedback conditions and therefore the continued, accurate control of the surgical system.

As used herein, the term “about” when used in connection with a referenced numeric indication means the referenced numeric indication plus or minus up to 10 percent of that referenced numeric indication. For example, the language “about 50” covers the range of 45 to 55. Similarly, the language “about 5” covers the range of 4.5 to 5.5.

The term “flexible” in association with a part, such as a mechanical structure, component, or component assembly, should be broadly construed. In essence, the term means the part can be repeatedly bent and restored to an original shape without harm to the part. Certain flexible components can also be resilient. For example, a component (e.g., a flexure) is said to be resilient if possesses the ability to absorb energy when it is deformed elastically, and then release the stored energy upon unloading (i.e., returning to its original state). Many “rigid” objects have a slight inherent resilient “bendiness” due to material properties, although such objects are not considered “flexible” as the term is used herein.

As used in this specification and the appended claims, the word “distal” refers to direction towards a work site, and the word “proximal” refers to a direction away from the work site. Thus, for example, the end of a tool that is closest to the target tissue would be the distal end of the tool, and the end opposite the distal end (i.e., the end manipulated by the user or coupled to the actuation shaft) would be the proximal end of the tool.

Further, specific words chosen to describe one or more embodiments and optional elements or features are not intended to limit the invention. For example, spatially relative terms—such as “beneath”, “below”, “lower”, “above”, “upper”, “proximal”, “distal”, and the like—may be used to describe the relationship of one element or feature to another element or feature as illustrated in the figures. These spatially relative terms are intended to encompass different positions (i.e., translational placements) and orientations (i.e., rotational placements) of a device in use or operation in addition to the position and orientation shown in the figures. For example, if a device in the figures is turned over, elements described as “below” or “beneath” other elements or features would then be “above” or “over” the other elements or features. Thus, the term “below” can encompass both positions and orientations of above and below: A device may be otherwise oriented (e.g., rotated 90 degrees or at other orientations) and the spatially relative descriptors used herein interpreted accordingly. Likewise, descriptions of movement along (translation) and around (rotation) various axes includes various spatial device positions and orientations. The combination of a body's position and orientation define the body's pose.

Similarly, geometric terms, such as “parallel”, “perpendicular”, “round”, or “square”, are not intended to require absolute mathematical precision, unless the context indicates otherwise. Instead, such geometric terms allow for variations due to manufacturing or equivalent functions. For example, if an element is described as “round” or “generally round,” a component that is not precisely circular (e.g., one that is slightly oblong or is a many-sided polygon) is still encompassed by this description.

In addition, the singular forms “a”, “an”, and “the” are intended to include the plural forms as well, unless the context indicates otherwise. The terms “comprises”, “includes”, “has”, and the like specify the presence of stated features, steps, operations, elements, components, etc. but do not preclude the presence or addition of one or more other features, steps, operations, elements, components, or groups.

Unless indicated otherwise, the terms apparatus, medical device, instrument, and variants thereof, can be interchangeably used.

Aspects of the invention are described primarily in terms of an implementation using a da Vinci® Surgical System, commercialized by Intuitive Surgical, Inc. of Sunnyvale, California. such as, for example, the da Vinci Xi® Surgical System (Model IS4000), and the da Vinci X® Surgical System (Model IS4200). Knowledgeable persons will understand, however, that inventive aspects disclosed herein may be embodied and implemented in various ways, including computer-assisted, non-computer-assisted, and hybrid combinations of manual and computer-assisted embodiments and implementations. Implementations on da Vinci® Surgical Systems (e.g., the Model IS4000, the Model IS2000, the Model IS1200) are merely presented as examples, and they are not to be considered as limiting the scope of the inventive aspects disclosed herein. As applicable, inventive aspects may be embodied and implemented in both relatively smaller, hand-held, hand-operated devices and relatively larger systems that have additional mechanical support.

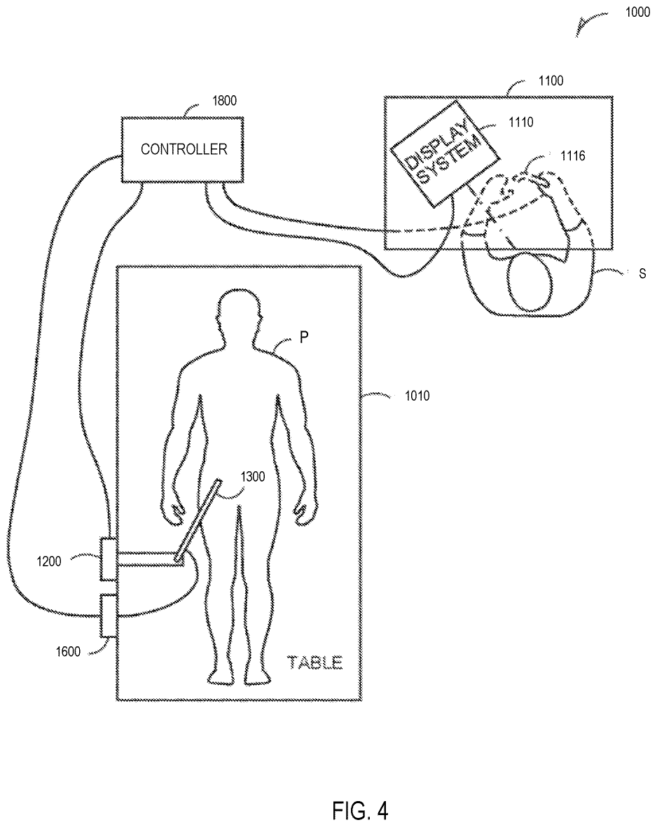

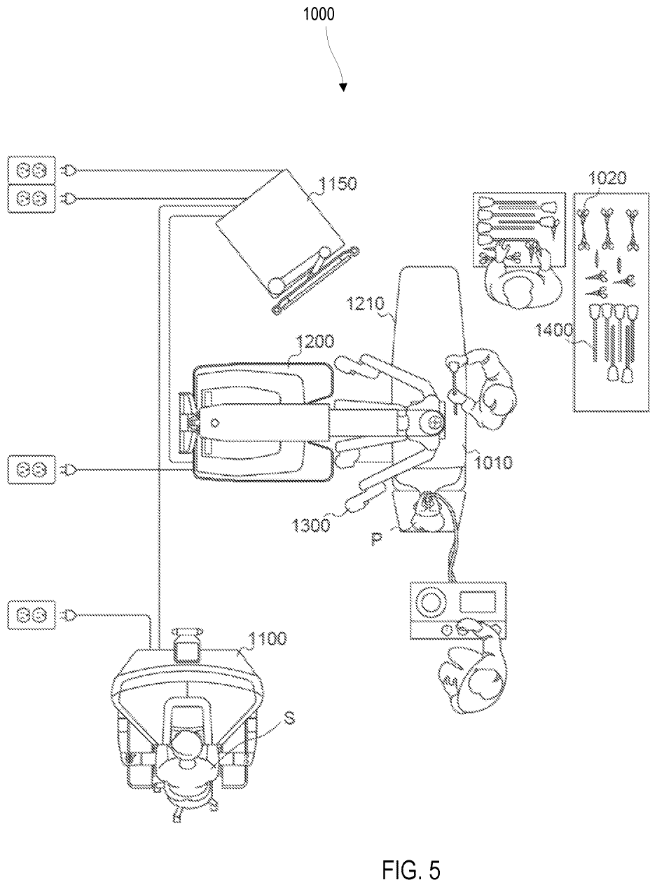

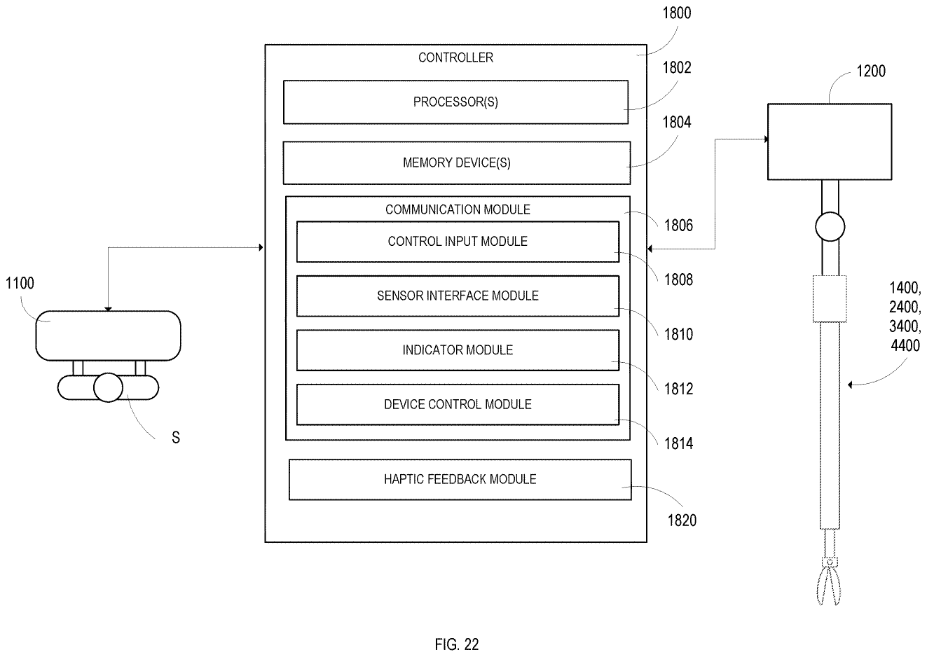

FIGS. 4 and 5 are plan view illustrations of a teleoperated surgical system 1000 that operates with at least partial computer assistance (a “telesurgical system”). Both telesurgical system 1000 and its components are considered medical devices. Telesurgical system 1000 may be a Minimally Invasive Robotic Surgical (MIRS) system used for performing a minimally invasive diagnostic or surgical procedure on a Patient P who is lying on an Operating table 1010. The system may have any number of components, such as a user control unit 1100 for use by a surgeon or other skilled clinician S (e.g., operator of the surgical system) during the procedure. The MIRS system 1000 can further include a manipulator unit 1200 (popularly referred to as a surgical robot) and an optional auxiliary equipment unit 1150. The manipulator unit 1200 can include an arm assembly 1300 and a surgical instrument tool assembly removably coupled to the arm assembly. The manipulator unit 1200 can manipulate at least one removably coupled instrument 1400 through a minimally invasive incision in the body or natural orifice of the patient P while the surgeon S views the surgical site and controls movement of the instrument 1400 through control unit 1100 with the assistance of a controller 1800. Further details of the controller 1800 are described below with reference to FIG. 22. An image of the surgical site is obtained by an endoscope 1600, such as a stereoscopic endoscope, which can be manipulated by the manipulator unit 1200 to orient the endoscope. The auxiliary equipment unit 1150 can be used to process the images of the surgical site for subsequent display to the Surgeon S via a display system 1110 of the user control unit 1100. The number of instruments 1400 used at one time will generally depend on the diagnostic or surgical procedure and the space constraints within the operating room, among other factors. If it is necessary to change one or more of the instruments 1400 being used during a procedure, an assistant removes the instrument 1400 from the manipulator unit 1200 and replaces it with another instrument 1400 from a tray 1020 in the operating room. Although shown as being used with the instruments 1400, any of the instruments described herein can be used with the MIRS 1000.

The user control unit 1100 is shown in FIGS. 4 and 5 as being in the same room as the patient so that the surgeon S can directly monitor the procedure, be physically present if necessary, and speak to an assistant directly rather than over the telephone or other communication medium. In other embodiments, however, the user control unit 1100 and the surgeon S can be in a different room, a completely different building, or other location remote from the patient, allowing for remote surgical procedures.

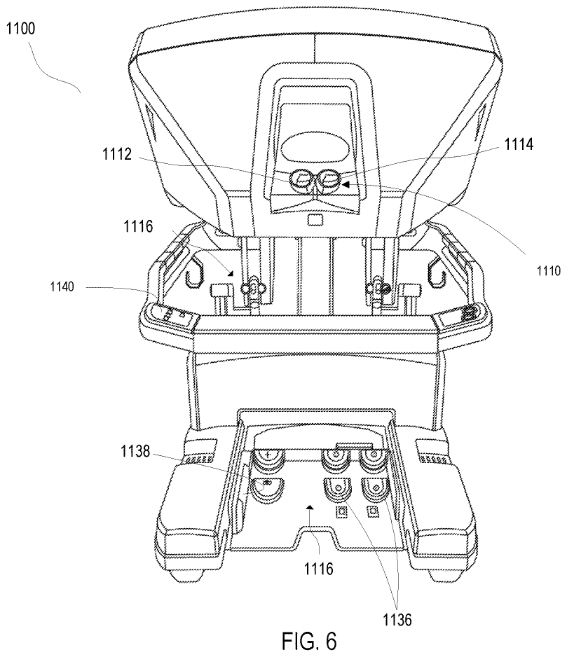

FIG. 6 is a perspective view of the control unit 1100. The user control unit 1100 may include one or more input control devices 1116 configured to be engaged by the surgeon S, which in turn cause the manipulator unit 1200 to manipulate one or more tools (e.g., medical device/surgical instrument). The input control devices 1116 provide at least the same degrees of freedom as instruments 1400 with which they are associated to provide the surgeon S with telepresence, or the perception that the input control devices 1116 are integral with (or are directly connected to) the instruments 1400. In this manner, the user control unit 1100 provides the surgeon S with a strong sense of directly controlling the instruments 1400. To this end, impressions (e.g., haptic feedback) of position, force, strain, or tactile feedback sensors (not shown) or any combination of such sensations, may be delivered from the instruments 1400 back to the surgeon S through the one or more input control devices 1116.

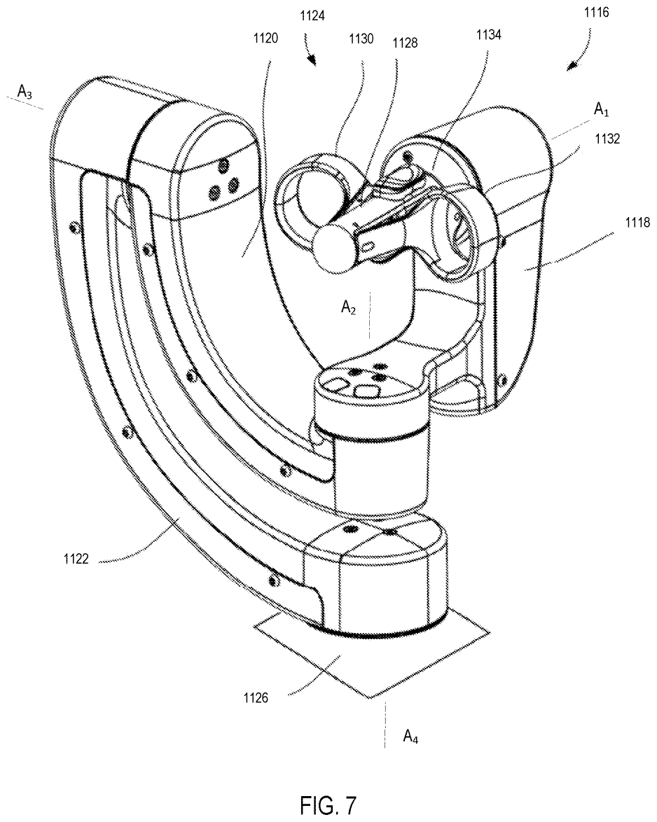

FIG. 7 shows a perspective view of an input control device 1116 configured for engagement via a portion of at least one hand of the surgeon S, according to an embodiment. In such a configuration, the input control device 1116 may include a first link 1118 (which functions as a first gimbal link), a second link 1120 (which functions as a second gimbal link), a third link 1122 (which functions as a third gimbal link), and an input handle 1124. The input control device 1116 may be mounted to a base portion 1126, which may be a part of a user control unit, such as the user control unit 1100 described herein. The input handle 1124 may include a handle portion 1128, a first handle input 1130, a second handle input 1132, and a handle input shaft 1134. In an embodiment, the handle input shaft 1134 may define a first rotational axis A₁ (which may function as a roll axis: the term roll is arbitrary) and may be rotatably coupled to the first link 1118. The handle portion 1128 is supported on the handle input shaft 1134 and is configured to be rotated relative to the first link 1118 about the first rotational axis A₁. The input shaft 1134 extends at least partially within the first link 1118. The first handle input 1130 and the second handle input 1132 can be manipulated to produce a desired action at the end effector (not shown). For example, in some embodiments, the first handle input 1130 and the second handle input 1132 can be squeezed together to produce a gripping movement at the end effector. The first and second handle inputs 1130, 1132 can be similar to the grip members shown and described in in U.S. Patent Application Pub. No. US 2020/0015917 A1 (filed Jun. 14, 2019), entitled “Actuated Grips for Controller,” which is incorporated herein by reference in its entirety for all purposes. For example, in other embodiments, however, the input handle 1124 need not include the handle inputs.

As depicted in FIG. 6, in an embodiment, at least one of the input control devices 1116 may be configured to be engaged via a portion of at least one foot of the surgeon S. In such a configuration, the input control device 1116 may include at least one pedal assembly 1136 and/or at least one foot-activated switch assembly 1138. Each pedal assembly 1136 and/or foot-activated switch assembly 1138 may include at least one switch (not shown) activated by the respective assembly. The switch(es) may be any suitable switch type, such as a toggle switch (toggling between opened and closed switch states), a normally open-momentarily closed switch, or a normally closed-momentarily open switch. Furthermore, the type of signal generated by the switch may be translated, encoded, or adapted to a proper voltage to control various types of medical equipment.

In an embodiment, the pedal assembly/foot-activated switch assembly 1136, 1138 may be assigned by the controller 1800 to control an operation of the instruments 1400. The functionality controlled by each pedal assembly/foot-activated switch assembly 1136, 1138 may be context sensitive and vary depending upon the type of instrument 1400 being controlled. For example, in an embodiment, the pedal assembly/foot-activated switch assembly 1136, 1138 may be assigned to control electrosurgical tools in response to one or more electrosurgical tools, such as a cautery implement. Accordingly, the surgical system 1000 may detect that one or more electrosurgical tools are mounted to the manipulator unit 1200 and may assign the appropriate control functions to the input control devices 1116 configured to be engaged via the portion of the foot of the surgeon S.

The user control unit 1100 may, in an embodiment, include one or more touchpads 1140 configured to receive an input from the surgeon S. The touchpad(s) 1140 may, for example, be a liquid crystal display (LCD) screen. The touchpad(s) 1140 may, as depicted in FIG. 6, be mounted in an arm-rest of the user control unit 1100. The surgeon S may utilize the touchpad(s) 1140 to access various operations, protocols, and/or settings of the surgical system 1000, such as user accounts, ergonomic settings, preferences, equipment configurations, operational status commands, and/or other similar processes. Additionally, the touchpad(s) 1140 may be utilized by the surgeon S to acknowledge various system messages, alerts, and/or warnings.

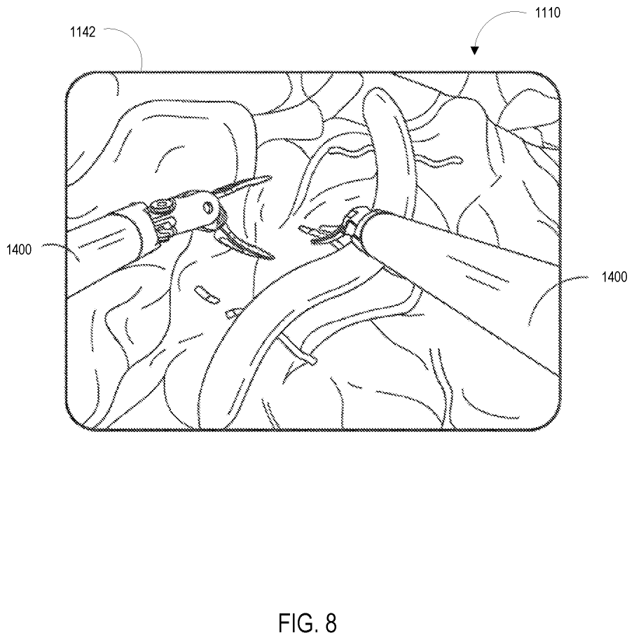

As further depicted in FIG. 6, the user control unit 1100 may include a display system 1110. As depicted in FIG. 8, the display system 1110 may define a field-of-view 1142 of the operator S. In an embodiment, the display system 1110 may include a left eye display 1112 and a right eye display 1114 for presenting the surgeon S with a coordinated stereoscopic view of the surgical site that enables depth perception. It should be appreciated that the surgical site may, for example, be within at least a portion of the body of the patient P.

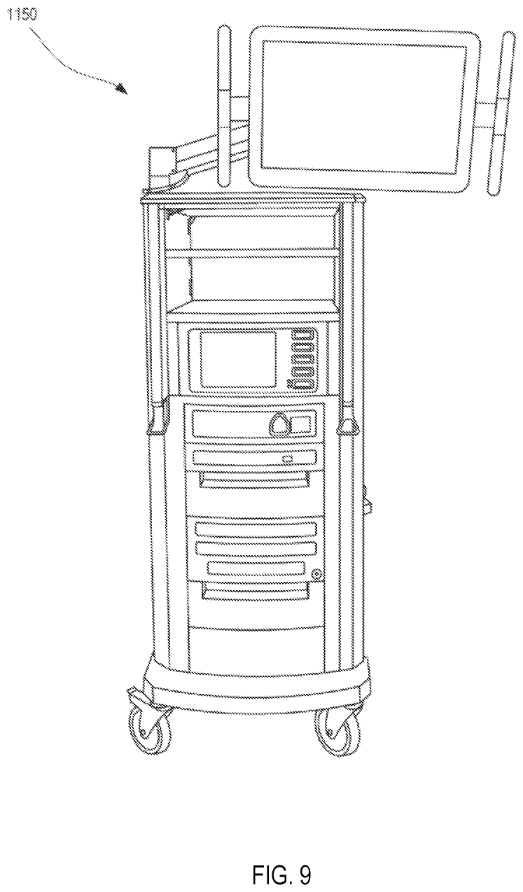

FIG. 9 is a perspective view of the auxiliary equipment unit 1150. The auxiliary equipment unit 1150 can be coupled with the endoscope 1600 and can include one or more processors to process captured images for subsequent display, such as via the display system 1110 of the user control unit 1100, or on another suitable display located locally (e.g., on the unit 1150 itself as shown, on a wall-mounted display) and/or remotely. For example, where a stereoscopic endoscope is used, the auxiliary equipment unit 1150 can process the captured images to present the surgeon S with coordinated stereo images of the surgical site via the left eye display 1112 and the right eye display 1114. Such coordination can include alignment between the opposing images and can include adjusting the stereo working distance of the stereoscopic endoscope. As another example, image processing can include the use of previously determined camera calibration parameters to compensate for imaging errors of the image capture device, such as optical aberrations.

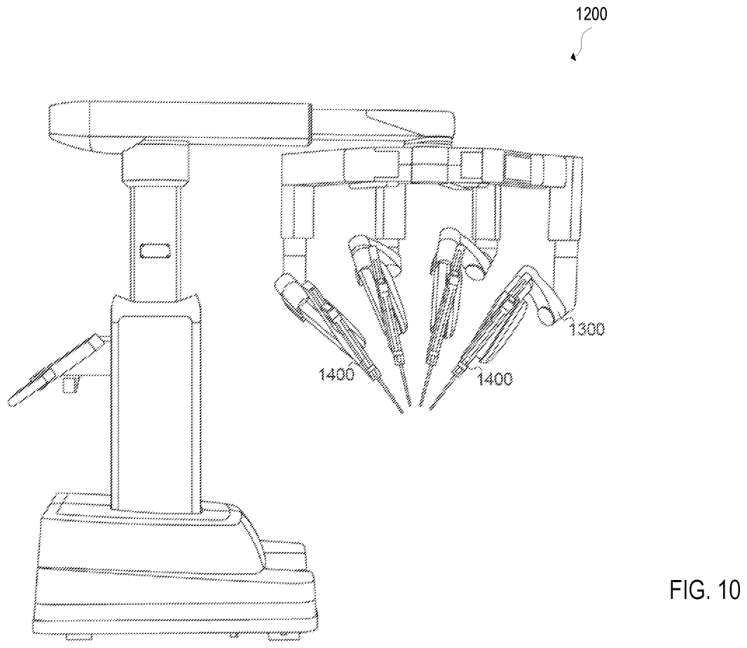

FIG. 10 shows a perspective view of the manipulator unit 1200. The manipulator unit 1200 includes the components (e.g., arms, linkages, motors, sensors, and the like) to provide for the manipulation of the instruments 1400 and an imaging device (e.g., the endoscope 1600) used for the capture of images of the site of the procedure. Specifically, the instruments 1400 and the imaging device can be manipulated by teleoperated mechanisms having one or more mechanical joints. Moreover, the instruments 1400 and the imaging device are positioned and manipulated through incisions or natural orifices in the patient P in a manner such that a center of motion remote from the manipulator and typically located at a position along the instrument shaft is maintained at the incision or orifice by either kinematic mechanical or software constraints. In this manner, the incision size can be minimized.

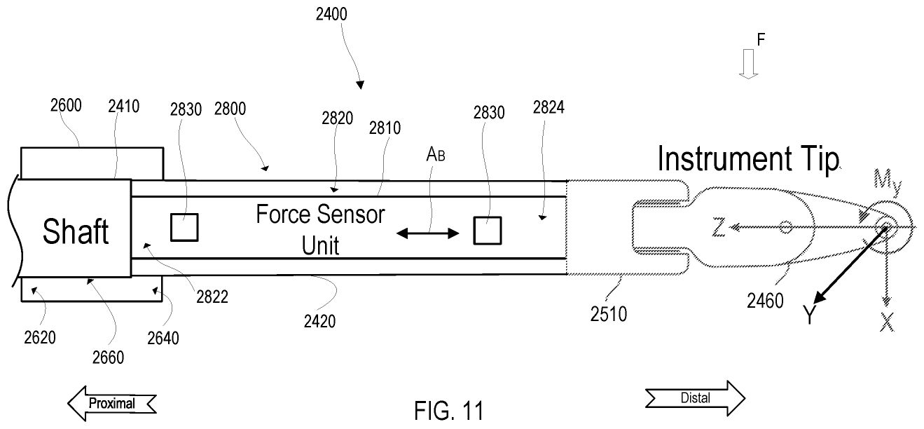

FIG. 11 is a schematic illustration of a distal end portion of a surgical instrument 2400, according to an embodiment. As depicted, a portion of the surgical instrument 2400 may be circumscribed by a cannula structure 2600. The cannula structure 2600 may have a proximal end 2620 and a distal end 2640. The cannula structure 2600 has a central channel 2660 that extends between the proximal end 2620 and the distal end 2640 through which the surgical instrument 2400 may be inserted during a medical procedure. The cannula structure 2600 may be a straight cannula as shown. However, in additional embodiments, the cannula structure 2600 may, for example, be a curved cannula having a combination of linear and nonlinear sections, a cannula with multiple non-parallel linear sections, a cannula with multiple curve sections having different characters, and/or a cannula with other combinations of linear and nonlinear sections.

The surgical instrument 2400 may include a shaft 2410, a force sensor unit 2800 including a beam 2810, with one or more strain sensors (e.g., strain gauges) 2830 mounted on a surface along the beam 2810. In some embodiments, a shroud 2420 may circumscribe at least a portion of the beam 2810, and an end effector 2460 may by coupled at a distal end portion 2824 of the surgical instrument 2400. The end effector 2460 can include, for example, articulatable jaws, a cautery instrument, and/or any other suitable surgical tool that is coupled to a link 2510 (e.g., a proximal clevis pin). In some embodiments, the link 2510 can be included within a wrist assembly having multiple articulating links. In some embodiments the link 2510 is included as part of the end effector 2460. The shaft 2410 may include a distal end portion 2412 that is coupled to a proximal end portion 2822 of the beam 2810. In some embodiments, the distal end portion 2412 of the shaft 2410 is coupled to the proximal end portion 2822 of the beam via another coupling component (such as an anchor or coupler, not shown). The shaft 2410 can also be coupled at a proximal end portion to a mechanical structure (not shown) configured to move one or more components of the surgical instrument, such as, for example, the end effector 2460.

In an embodiment, the beam 2810 may include a middle portion 2820 (which functions as an active portion of the beam for force sensing), a proximal end portion 2822 and a distal end portion 2824. The beam 2810 defines a beam center axis A_{B}, which can be aligned within a center axis (not shown) of the instrument shaft 2410. The distal end portion 2824 of the beam 2810 may be coupled to the end effector 2460 via a link 2510. In some embodiments, the link 2510 can be, for example, a clevis of the end effector 2460. It should be appreciated that the beam 2810 can include any number of strain sensors 2830 in various arrangements.

Generally, during a medical procedure, the end effector 2460 contacts anatomical tissue, which may result in X, Y, or Z direction forces being imparted on the end effector 2460 and that may result in moment forces such as a moment My about a y-direction axis as shown in FIG. 11. The strain sensor(s) 2830, which can be a strain gauge, can measure strain in the beam 2810 which can be used to determine the forces imparted on the end effector 2460 in the X and Y axes directions. These X and Y axes forces are transverse (e.g., perpendicular) to the Z axis (which is parallel or collinear with the center axis A_{B}). Such transverse forces acting upon the end effector 2460 can cause a bending of the beam 2810 (about either or both of the X axis or the Y axis), which can result in a tensile strain imparted to one side of the beam 2810 and a compression strain imparted to the opposite side of the beam 2810. The strain sensors 2830 on the beam 2810 may measure such tensile and compression strains. It should be appreciated that the output of the force sensor unit 2800 may be utilized by a controller, such as the controller 1800 of system 1000 described above, to determine the haptic feedback to deliver to the surgeon S via the input control device(s) 1116.

Although shown as including only the force sensor unit 2800, in some embodiments, the instrument 2400 (or any of the instruments described herein) can include additional force sensor units to measure the axial force(s) (i.e., in the direction of the Z-axis parallel to the beam center axis A_{B}) imparted on the end effector 2460. An axial force sensor unit in an example surgical instrument can comprise a deflectable planar diaphragm sensor that deflects in response to a force. Alternatively, a deflectable ferrite core can be used within an inductive coil may be used or a or a fiber Bragg grating formed within an optical fiber can be used, for example. Other axial force sensor units may be used to sense a resilient axial displacement of the shaft 2410 (e.g., relative to the proximally mounted mechanical structure, not shown). An axial force Fz imparted to the end effector 2460 can cause axial displacement of the shaft 2410 in a direction along a center axis of the shaft (substantially parallel to the beam center axis A_{B}). The axial force Fz may be in the proximal direction (e.g., a reactive force resulting from pushing against tissue with the end effector) or it may be in the distal direction (e.g., a reactive force resulting from pulling tissue grasped with the end effector).

In an embodiment, X and Y forces imparted on the end effector 2460 may result in strain in the beam 2810 when the beam 2810 is displaced (e.g., bent) relative to the center axis A_{B} of the beam 2810, and thus relative to a center axis of the shaft 2410. In other words, a distal end portion 2824 of the beam 2810 can bend relative to a proximal end portion 2822 of the beam 2810 such that the end portion 2824 of the beam 2810 is displaced a deflection distance relative to the center axis A_{B}.

In some embodiments, the shroud 2620 and/or the cannula structure 2600 can limit the displacement of the beam 2810 and produce a reactive moment therein. In an embodiment wherein the displacement of beam 2810 is limited by the shroud 2620 and/or the cannula structure 2600, the strain distribution over the length of the beam 2810 may deviate relative to displacements of the beam 2810 that are not limited. As a result, the strain sensor(s) 2830 may produce signals that do not accurately represent the force F applied to the end effector 2460. For example, the signals from the strain sensor(s) 2830 may indicate a force that is decreasing when, the force acting on the end effector 2460 is actually increasing (e.g., a force inversion condition may exist). Insofar as the controller 1800 may utilize the signals from the strain sensor(s) 2830 to generate the haptic feedback delivered to the surgeon S, the inaccurate representation of the force F resulting in inaccurate haptic feedback may be undesirable. It should therefore be appreciated that mitigating the impact of inaccurate haptic feedback may be beneficial to the operation of the surgical system 1000.

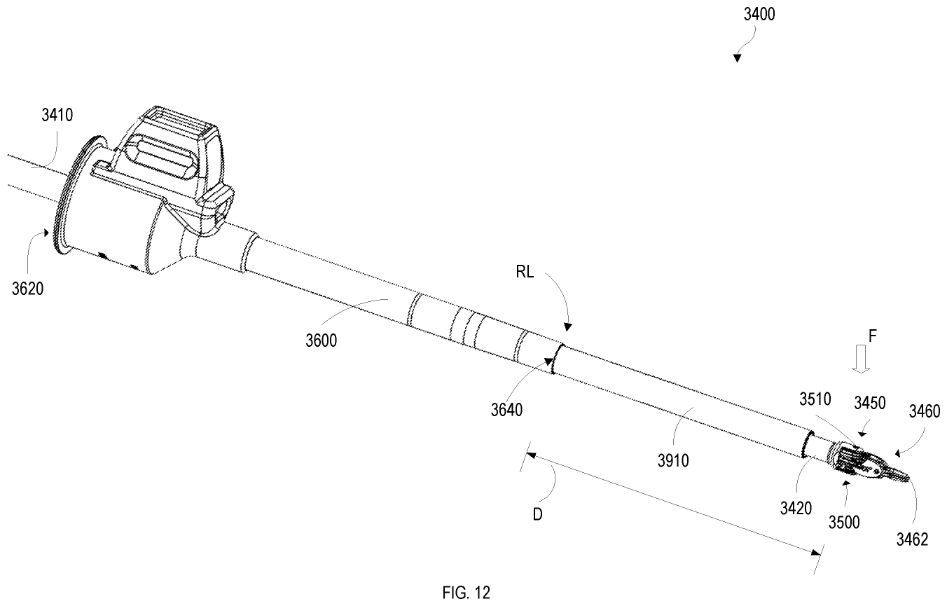

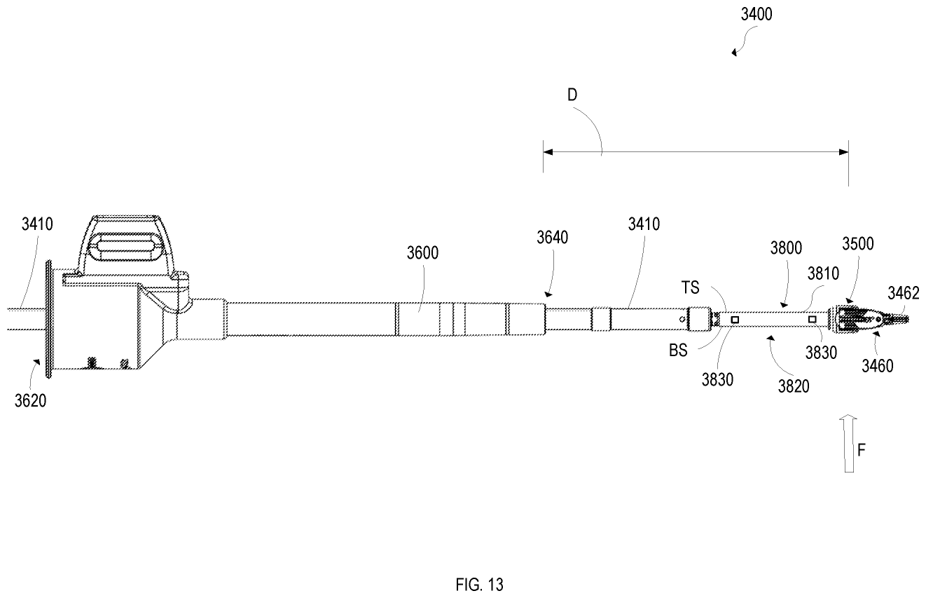

FIGS. 12 and 13 depict a perspective view and a side view (with the outer shaft and shroud removed for clarity) of a medical instrument 3400 and a cannula 3600 according to an embodiment. In some embodiments, the instrument 3400 or any of the components therein are optionally parts of a surgical system that performs surgical procedures. The surgical system may include a manipulator unit, a series of kinematic linkages, a series of cannulas, or the like. The instrument 3400 (and any of the instruments described herein) can be used in any suitable surgical system, such as the MIRS system 1000 shown and described above.

The instrument 3400 may include a proximal mechanical structure (not shown), an outer shaft 3910, a shaft 3410, a force sensor unit 3800 that includes a beam 3810, a wrist assembly 3500, and an end effector 3460. As depicted in FIG. 12, in an embodiment, a shroud 3420 may circumscribe at least a portion of the beam 3810. Although not shown, the instrument 3400) can also include a number of cables that couple the mechanical structure to the wrist assembly 3500 and end effector 3460. The instrument 3400 is configured such that select movements of the cables produces rotation of the wrist assembly 3500 (i.e., pitch rotation) about an axis of rotation (which functions as a pitch axis, the term pitch is arbitrary), yaw rotation of the end effector 3460 about an additional axis of rotation (which functions as the yaw axis, the term yaw is arbitrary), a cutting rotation of the tool members of the end effector 3460, or any combination of these movements. Changing the pitch or yaw of the instrument 3400 can be performed by manipulating the cables in a similar manner as described, for example, in U.S. Patent No. U.S. Pat. No. 8,821,480 B2 (filed Jul. 16, 2008), entitled “Four-Cable Wrist with Solid Surface Cable Channels,” which is incorporated herein by reference in its entirety.

In an embodiment, the end effector 3460) can include at least one tool member 3462 having a contact portion configured to engage or manipulate a target tissue during a surgical procedure. For example, in some embodiments, the contact portion can include an engagement surface that functions as a gripper, cutter, tissue manipulator, or the like. In other embodiments, the contact portion can be an energized tool member that is used for cauterization or electrosurgical procedures. The end effector 3460 may be operatively coupled to the mechanical structure such that the tool member 3462 rotates relative to shaft 3410. In this manner, the contact portion of the tool member 3462 can be actuated to engage or manipulate a target tissue during a surgical procedure. The tool member 3462 (or any of the tool members described herein) can be any suitable medical tool member. Moreover, although only one tool member 3462 is identified, as shown, the instrument 3400 can include two tool members that cooperatively perform gripping or shearing functions. In other embodiments, an end effector can include more than two tool members.

In an embodiment, the force sensor unit 3800 may include one or more strain sensors 3830 mounted on the beam 3810. The strain sensors 3830 can be, for example, strain gauges, and may be used to measure forces imparted on the surgical instrument during a surgical procedure as described in more detail below. In an embodiment, the beam 3810 may define at least three side surfaces disposed acutely to each other. In an additional embodiment, the beam 3810 may define at least four side surfaces disposed perpendicular to one another. The strain sensors 3830 may be mounted to the side surfaces in appropriate locations. The force sensor unit 3800 is communicatively coupled to a controller of the surgical system (e.g., the controller 1800 of FIG. 22).

In use, the end effector 3460 may contact anatomical tissue, which may result in X, Y, or Z direction forces (see FIG. 11) being imparted on the end effector 3460. This contact may also result in moment forces about the various axes. The strain sensors 3830 may be used to measure strain in the beam 3810 as a result of such forces imparted on the end effector 3460. More specifically, the strain sensors 3830 can measure forces imparted on the end effector 3460 that are transverse (e.g., perpendicular) to a center axis of the beam 3810 as such forces are transferred to the beam 3810 in the X and Y directions (see FIG. 11). Specifically, the transverse forces acting upon the end effector 3460 can cause a slight bending of the beam 3810, which can result in a tensile strain imparted to one side of the beam 3810 and a compression strain imparted to an opposing side of the beam 3810. The strain sensors 3830 may be coupled to the beam 3810 to measure such tensile and compression forces, with the resultant measurements being communicated to the controller.

More specifically, when a force F is imparted on a distal portion of the medical device 3400 (e.g., at end effector 3460) in the X or Y directions (see FIG. 11 for reference to X, Y and Z directions), such transverse force can cause the beam 3810 to bend (about either or some combination of the X axis or the Y axis), which can result in a tensile strain imparted to one side of the beam 3810 and a compression strain imparted to the opposite side of the beam 3810. The strain sensors 3830 on the beam 3810 can measure such tensile and compression strains. For example, when a force F is applied to the end effector 3460, the beam 3810 alone or in combination with the shroud 3420 may bend downward in the direction of the force F, such that a tensile strain would be imparted on a top side TS of the beam 3810 and a compression strain would be imparted on a bottom side BS of the beam 3810.

The cannula 3600 and/or the shroud 3420 may have a stiffness that is greater than a stiffness of the beam 3810. Thus, in some embodiments, contact between beam 3810 and the shroud 3420 and/or the cannula 3600 may limit the displacement of the beam 3810 and result in the production of a reactive moment within the beam 3810. In an embodiment wherein the displacement of beam 3810 is limited by the shroud 3620 and/or the cannula 3600, the strain distribution over the length of the beam 3810 may deviate relative to displacements of the beam 3810 that are not limited. As a result, the strain sensor(s) 3830 may produce signals that do not accurately represent the force F applied to the end effector 3460. In an embodiment, the signals from the strain sensor(s) 3830 may indicate a force that is decreasing when, the force F acting on the end effector 3460 is actually increasing (e.g., a force inversion condition may exist).

For example, in an embodiment, the distance D corresponds to an insertion distance of a designated portion 3450 of the instrument 3400 relative to the cannula 3600. The designated portion 3450 may, for example, be at a proximal clevis pin 3510 of the wrist assembly 3500. As shown, the distance D is determined between the designated portion 3450 and a defined reference location RL on the cannula 3600 (e.g., the distal end 3640). At certain distances, (e.g., insertion distances) the contact between the beam 3810 and the shroud 3420 and/or the cannula 3600 may occur when the beam 3810 bends in response to the force F. For example, in an embodiment where the distance D may be less than 25 mm (e.g., 17 mm or less), the bending of the beam 3810 may result in the contact and, therefore, may result in a force inversion condition. The below described methods of control of a surgical system provide various operational controls of the haptic feedback provided to a user of a medical instrument when such a condition occurs.

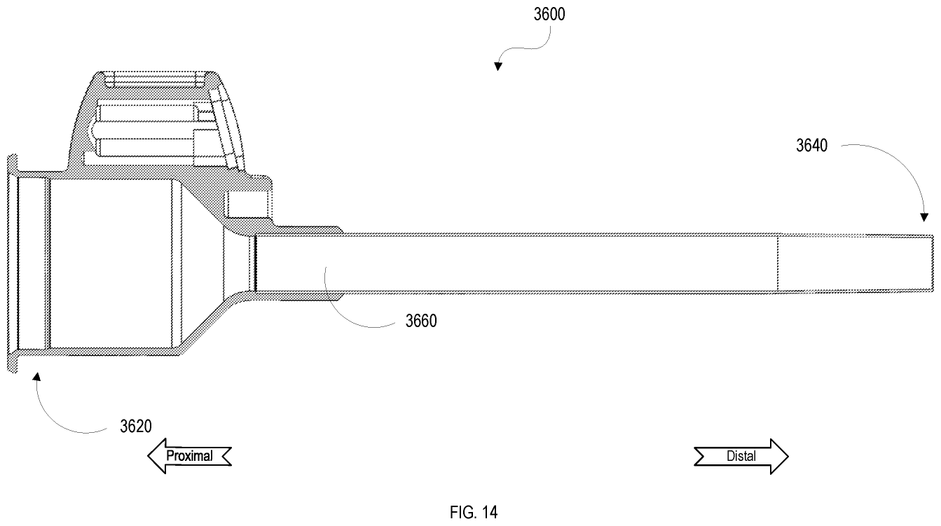

FIG. 14 depicts a cross-sectional view of the cannula 3600 without the instrument 3400 inserted therein. As depicted, the cannula 3600 may be configured to circumscribe at least a portion of the instrument 3400 so as to facilitate access of the surgical site by the end effector 3460. Accordingly, the cannula 3600 may have a proximal end 3620 and a distal end 3640. A central channel 3660 may extend between the proximal and distal ends 3620, 3640. As such, the cannula 3600 may form a channel or passage through which the instrument 3400) may be inserted to access the surgical site. As depicted, the cannula 3600 may be a straight cannula. Additionally, the cannula 3600 may have a stiffness that is greater than a stiffness of the beam 3810 and/or the shroud 3420.

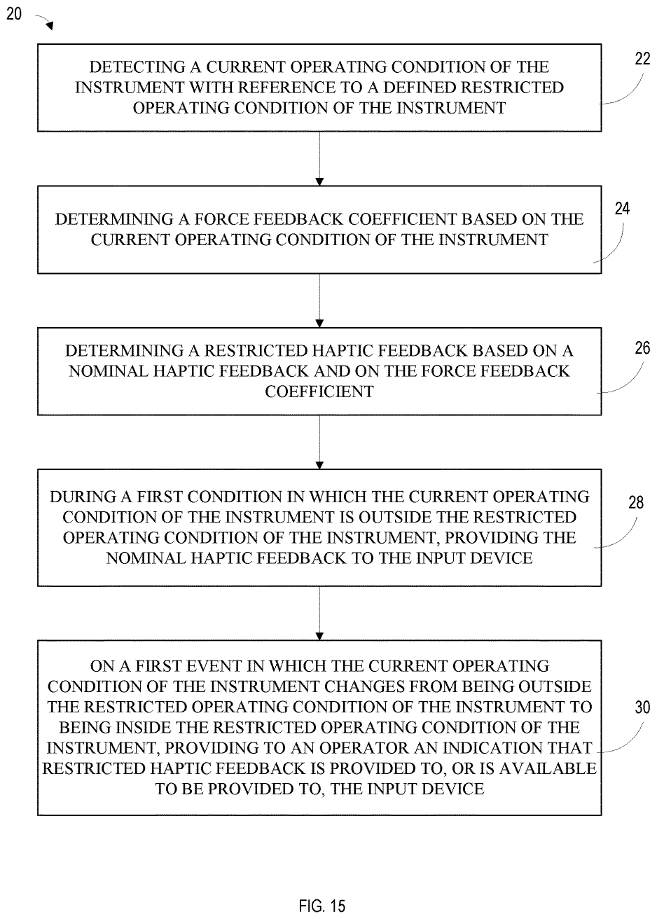

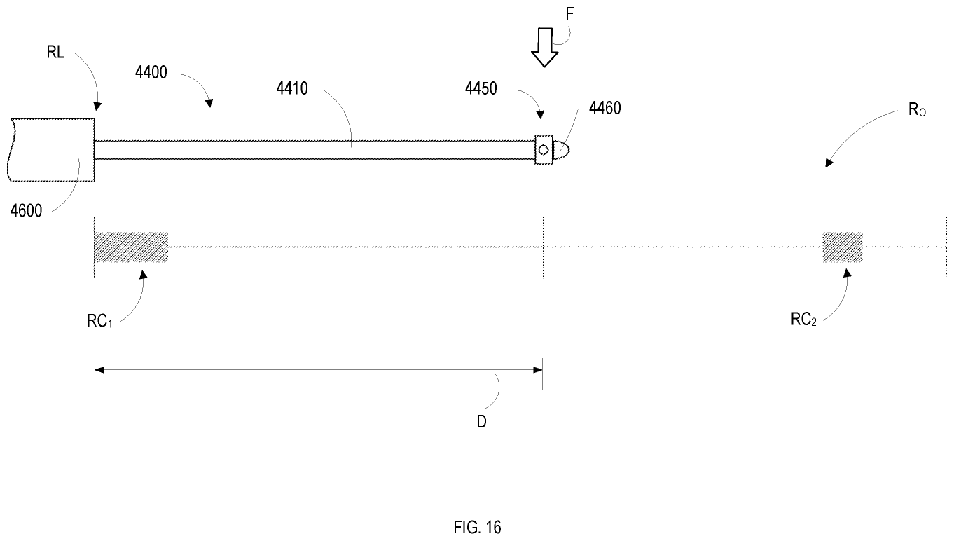

FIG. 15 is a flow chart of a method 20 of control for a surgical system according to an embodiment. The method 20 may, in an embodiment, be performed via a teleoperated system, such as system 1000 as described with reference to FIGS. 4-10 and FIG. 22. However, it should be appreciated that in various embodiments, aspects of the method 20 may be accomplished via additional embodiments of the system 1000 or components thereof, such as instrument 2400, instrument 3400, and/or instrument 4400 as described herein. Accordingly, the method 20 may be implemented on any suitable device as described herein. FIGS. 16-18 are schematic illustrations of a medical instrument 4400 showing various operating conditions for a medical instrument, such as medical instruments 1400, 2400, and/or 3400 described herein. The medical device 4400 includes a shaft 4410 coupled to an end effector 4460, and is shown extending distally from a cannula 4600. The method 20 may be implemented, at least in part, via the controller 1800 of the system 1000 (as described in FIGS. 4-10 and FIG. 22). Thus, the method 20 is described below with reference to medical instrument 4400) and the controller 1800 of the system 1000 depicted in FIGS. 4-10 and FIG. 22, but it should be understood that the method 20 can be employed using any of the medical devices/instruments and controllers described herein.

As depicted at 22 in FIG. 15, controller 1800 detects a current operating condition of the medical instrument 4400. The current operating condition of the instrument 4400 may be determined with reference to a defined restricted operating condition of the instrument 4400, as described in more detail below.

As depicted in FIG. 16, in some embodiments, the restricted operating condition of the instrument 4400 may be at least one portion (depicted as shaded regions RC₁ and RC₂) of an operating range R_{O} of the medical instrument 4400. The portion(s) may be a segment of the operating range R_{O} of the medical instrument 4400 in which a determination by the controller 1800 of a force acting on the instrument 4400 may deviate from the force that is actually acting on the instrument 4400. The operating range R_{O} may, for example, correspond to a range of possible insertion distances of the instrument 4400 defined between a minimal insertion distance (e.g., a point of emergence from a cannula 4600) and a maximal insertion distance. The maximal insertion distance may, for example, be mechanically limited by a length of the shaft 4410 (e.g., shaft 2410 and/or shaft 3410) or other component of the instrument 4400.

Accordingly, to detect the current operating condition of the instrument 4400, the controller 1800 may, in an embodiment, determine a distance D between a designated portion 4450 of the instrument 4400 and a defined reference location RL on the cannula 4600 (e.g., cannula 2600 and/or cannula 3600) of the surgical system 1000. For example, the designated portion 4450) may correspond to a component of a wrist assembly (e.g., wrist assembly 3500) of the medical instrument 4400, such as a point of rotation (e.g., the linkage 2510) and/or the proximal clevis pin 3510), but may, in additional embodiments, correspond to other suitable features of the instrument 4400. The defined reference location RL on the cannula 4600 may, for example, correspond to a distal end (e.g., distal end 3640) of the cannula 4600.

By way of illustration, in an embodiment, the restricted operating condition may correspond to the portion of the operating range R_{O} identified by the shaded region RC₁. In such an embodiment, the restricted operating condition may correspond to a distance D (e.g., an insertion distance) that is 25 mm or less (e.g., 17 mm or less). When the distance D is 25 mm or less, a force F acting on the instrument 4400 may result in a contact between a portion of the instrument 4400 and the cannula 4600. Insofar as the cannula may be substantially stiff, this contact may result in at least one sensor (e.g., the strain sensor(s) 2830) producing signals that do not accurately represent the force F. For example, the signals from the sensor(s) (e.g., as received by a sensor interface module 1810 shown in FIG. 22) may indicate a force that is decreasing when, the force F is actually increasing. As a result, a force inversion condition may exist. Insofar as the controller 1800 may utilize the signals from the sensor(s) to generate the haptic feedback delivered to the surgeon S, the inaccurate representation of the force F resulting in inaccurate haptic feedback may be undesirable. It should therefore be appreciated that minimizing a reliance on and/or utilization of the strain sensor signals when the distance D is 25 mm or less, while otherwise maintaining the operative capability of the instrument 4400, may be desirable.

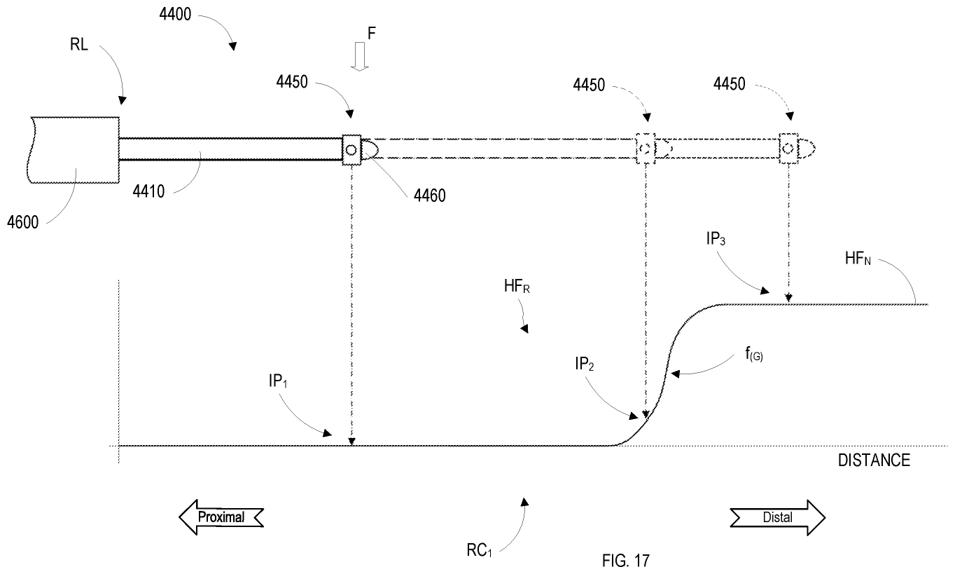

As depicted at step 24 in FIG. 15, the controller 1800 may determine a force feedback coefficient (e.g., via the haptic feedback module 1820 (FIG. 22)) based on the current operating condition of the instrument 4400. The force feedback coefficient may facilitate the correlation of a delivered haptic feedback magnitude/level to the force F acting on the instrument 4400. Said another way, the force feedback coefficient is a value within the range of zero to one that adjusts the delivered haptic feedback. In an embodiment, a force feedback coefficient equal to one corresponds to a nominal haptic feedback HF_{N} (FIG. 17). In other words when the force feedback coefficient is equal to one, the delivered haptic feedback is the same as the nominal haptic feedback that is based on the sensed value of the force F (i.e., the haptic feedback is not restricted or reduced). Similarly, in an embodiment, a force feedback coefficient of less than one correspond to a restricted haptic feedback HF_{R} (FIG. 17). When the force feedback coefficient is zero, the restricted haptic feedback HF_{R} corresponds to a disabled haptic feedback condition of the surgical system 1000. As such, the restricted haptic feedback HF_{R} may, as depicted at step 26, be determined based on the nominal haptic feedback HF_{N} and the force feedback coefficient. Thus, the force feedback coefficient facilitates the delivery of a level of haptic feedback to the surgeon S that is a percentage of the nominal haptic feedback HF_{N}, including delivering zero percent of the nominal haptic feedback (e.g., disabling, at least temporarily, the haptic feedback system).

In some embodiments, the restricted haptic feedback HF_{R} corresponds to a full restriction of the nominal haptic feedback HF_{N}. In such embodiments, the magnitudes of the restricted haptic feedback HF_{R} along each of the x-axis, the y-axis, and the z-axis (see e.g., FIG. 11) are each less than the corresponding nominal haptic feedback HF_{N} magnitudes. However, in some embodiments, the restricted haptic feedback HF_{R} corresponds to a partial restriction of the nominal haptic feedback HF_{N}. For example, in such embodiments, the magnitudes of the restricted haptic feedback HF_{R} along of the x-axis and the y-axis may be less than the corresponding nominal haptic feedback HF_{N} magnitudes while magnitudes along the z-axis are unaffected.

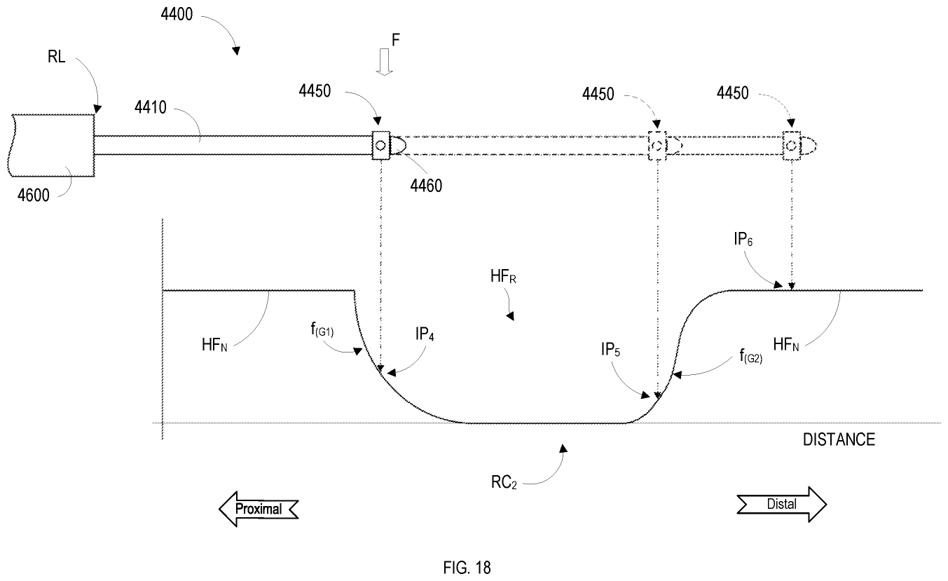

In some embodiments, such as depicted in FIGS. 17 and 18, the force feedback coefficient may be defined by the controller 1800 (e.g., via the haptic feedback module 1820) based on a gain function f_{(G)} and/or a saturation function. The gain function f_{(G)} or the saturation function may define a curve (e.g., a feedback-transition curve) descriptive of a rate of change of the force feedback coefficient relative to a variable, such as the distance D and/or a maximal haptic force. In this manner, the method can produce a smooth transition between producing the nominal haptic feedback HF_{N} and disabling the haptic feedback. In an embodiment, such as depicted in FIG. 17, the gain function f_{(G)} may describe a sigmoid curve. For example, when the designated portion 4450 of the instrument 4400 is at position IP₂, the force feedback coefficient may be defined by the gain function f_{(G)} and/or the saturation function (e.g., may lie on the sigmoid curve as illustrated).

In some embodiments employing the gain function f_{(G)} to modify the nominal haptic feedback HF_{N}, the controller 1800 will multiply the sensed force (e.g., as indicated by the output of the force sensor unit 2800 or the force sensor unit 3800) by a factor that is a function of a variable, such as the distance D. However, in some embodiments a saturation function can be employed to modify the nominal haptic feedback HF_{N}, by the multiplication of a maximal haptic feedback magnitude by a factor that is a function of a variable, such as distance D and the magnitude of the sensed force ((e.g., as indicated by the output of the force sensor unit 2800 or the force sensor unit 3800). In other words, the saturation function may be employed to reduce a maximal force that may be delivered by the haptic feedback system to the operator S to a fraction of a nominal maximal haptic feedback magnitude.

By way of additional illustration, in an embodiment, the gain function f_{(G)} may describe a linear ramp. However, in an embodiment, such as depicted in FIG. 18, the gain function f_{(G)} may describe an exponential curve. For example, when the designated portion 4450 of the instrument 4400 is at position IP₄, the force feedback coefficient may be defined by the gain function f_{(G1)} (e.g., may lie on the exponential curve as illustrated). In further embodiments, the gain function f_{(G)} and/or the saturation function may define other suitable curves. It should thus be appreciated that the nominal haptic feedback HF_{N} may be modified by the force feedback coefficient in accordance with the gain function f_{(G)} so that a portion may be provided to the surgeon S when the instrument 4400 encounters the corresponding variable (e.g., is at a corresponding distance D).

To define the force feedback coefficient based on the gain function f_{(G)} and/or the saturation function, the controller 1800 may determine the gain function f_{(G)} and/or the saturation function based on an operating condition of the instrument 4400 or impacting the instrument 4400. In an embodiment, the operating condition may correspond to an acceleration of the designated portion 4450 of the instrument 4400. For example, when the designated portion 4450 has a relatively high acceleration, a relatively high rate of change of the force feedback coefficient may be desirable in order to affect a relatively rapid change in the haptic feedback. However, when the designated portion 4450 has a relatively/low acceleration, a relatively low rate of change of the force feedback coefficient may be desirable in order to affect a more gradual change in the haptic feedback than would be observed at a higher rate of change of the force feedback coefficient. In additional embodiments, the gain function f_{(G)} and/or the saturation function may be defined by an electrical power generated by the surgical system (e.g., for a cautery instrument) and/or a direction of a change in force. the gain function f_{(G)} and/or the saturation function may be tailored to achieve a change in the haptic feedback at a rate that is most beneficial for the operating condition of the instrument 4400.

In an embodiment, different gain/saturation functions f_{(G)} may be utilized at different distances D of the designated portion 4450 of the instrument 4400. For example, as depicted in FIG. 18, when the designated portion 4450 is at position IP₄, the force feedback coefficient may be defined by a first gain function f_{(G1)} (e.g., an exponential curve). However, when the designated portion 4450 is at position IP₅, the force feedback coefficient may be defined by a second gain function f_{(G2)} (e.g., a sigmoid curve). This can allow one function to control the rate of restricting (ramping down) the haptic feedback and a second, different function to control the rate of returning from a restricted feedback condition to full haptic feedback.

Referring to step 28 in FIG. 15, in a first condition, the current operating condition of the instrument 4400 may be outside the restricted operating condition (e.g., positions IP₃ and IP₆) of the instrument 4400. Further, at step 28, when the current operating condition is outside the restricted operating condition, the controller 1800 (e.g., via the haptic feedback module 1820) may provide the nominal haptic feedback HF_{N} to the input device 1116 of the system 1000 (as described in FIGS. 4-10 and FIG. 22). The nominal haptic feedback HF_{N} may correspond to a designed/intended haptic feedback characteristic and/or magnitude delivered to the surgeon S when a restriction on the operating condition of the instrument 4400 is absent. Providing the nominal haptic feedback HF_{N} may include establishing the force feedback coefficient at a value equal to one such that 100% of the design/intended haptic feedback is provided to the surgeon S.

As depicted at step 30, on a first event, the current operating condition of the instrument 4400) may change/transition from being outside the restricted operating condition (e.g., positions IP₃ and IP₆) to being inside the restricted operating condition (e.g., positions IP₁, IP₂, IP₄, and IP₅) of the instrument 4400. Upon detecting such a change/transition, the controller 1800 may provide the operator/surgeon S an indication that restricted haptic feedback is being provided, or is available to be provided, to the input device 1116 of the system 1000. In other words, on the first event, the operator/surgeon S may receive an indication that the haptic feedback provided to the input device 1116 is deviating, or will deviate, from the nominal haptic feedback HF_{N}.

It should be appreciated that the first event may result from a repositioning of the designated portion 4450 of the instrument 4400 (e.g., via the device control module 1814 (FIG. 22)). For example, in an embodiment, such as depicted in FIG. 18, proximal movement of the instrument 4400 may result in the designated portion 4450 transitioning from position IP₆ (e.g., outside the restricted operating condition) to position IP₅ (e.g., inside the restricted operating condition). However, in an additional embodiment, the first event may be coincident with the emergence of the designated portion 4450 from the defined reference location RL on the cannula 4600, such as may occur during the insertion of the instrument 4400 into the patient P or withdrawal of the instrument 4400 from the patient. In such an embodiment, the instrument 4400 may remain in the restricted operating condition until the designated portion 4450 has been advanced a sufficient distance D.

In some embodiments, a second condition may exist when the current operating condition (e.g., distance D of the designated portion 4450 from the defined reference location RL) of the instrument 4400 corresponds to a positioning of the instrument 4400 inside of the restricted operating condition of the instrument 4400. When the second condition is present, the controller 1800 (e.g., via the haptic feedback module 1820) may provide the restricted haptic feedback HF_{R} to the input device 1116. In other words, whenever the current operating condition of the instrument 4400 is inside the restricted operating condition, the nominal haptic feedback HF_{N} may be reduced and/or disabled. In an embodiment, the delivery of the restricted haptic feedback HF_{R} may be accomplished automatically (e.g., without input of an operator S shown in FIG. 22) upon notification to the operator/surgeon S (e.g., via the indicator module 1812 (FIG. 22)). For example, in an embodiment, the controller 1800 may be configured to automatically provide the restricted haptic feedback HF_{R} to the input device 1116 upon insertion of the instrument and maintain the restricted haptic feedback HF_{R} until the designated portion 4450) has advanced a sufficient distance D.

In an additional embodiment wherein the second condition may be present, before the restricted haptic feedback HF_{R} may be provided, an acknowledging input from the operator S may be required. In such an embodiment, the input by the operator/surgeon S may correspond to a second event. During the second event, an input by the operator/surgeon S may acknowledge the indication of the restricted feedback. This acknowledgment may be received at the controller 1800 (e.g., via the control input module 1808 (FIG. 22)). In such an embodiment, the controller 1800 (e.g., via the haptic feedback module 1820) may provide the restricted haptic feedback HF_{R} to the input device 1116 (e.g., to the operator/surgeon S). In other words, in some embodiments, the nominal haptic feedback HF_{N} may not be modified without the acknowledging input being received by the controller 1800 from the operator/surgeon S. It should be appreciated that requiring the acknowledging input may facilitate a recognition by the operator/surgeon S that a magnitude of the haptic feedback that will be felt will be less than the magnitude of the haptic feedback that would otherwise be anticipated when performing a similar action/operation outside the restricted operating condition.

In some embodiments, providing the nominal haptic feedback HF_{N} includes generating the nominal haptic feedback HF_{N} based on a first strain sensor signal from the instrument 4400 (e.g., such as may be received from the force sensor unit 2800 or the force sensor unit 3800) received at the controller 1800.

Providing the restricted haptic feedback HF_{R} may include providing a haptic feedback level that is less than a nominal haptic feedback level. In other words, the magnitude/intensity of the haptic feedback provided to the surgeon S may be less than 100% of the nominal haptic feedback level. The reduction in the haptic feedback level provided to the surgeon S may result from a feedback coefficient that is less than one. In an embodiment, the haptic feedback level may be reduced to zero in order to disable the haptic feedback system of the surgical system 1000 when the current operating condition of the instrument 4400 is within the restricted operating condition. Accordingly, in an embodiment wherein the haptic feedback system is disabled, the outputs of the force sensor unit are filtered/muted so as to preclude utilization thereof so long as the designated portion 4450 of the instrument 4400 is within the restricted operating condition.

The indication that the restricted haptic feedback HF_{R} is being provided or is available may be a visual indication, a haptic indication, and/or an audible indication. For example, the controller 1800 may, in an embodiment, be configured to generate a graphical indication of a deviation of the restricted haptic feedback HF_{R} from the nominal haptic feedback HF_{N} (e.g., via the indicator module 1812). The controller 1800 may maintain (e.g., via the display system 1110) the graphical indication within a field-of-view 1142 (see FIG. 8) of the operator/surgeon S when the instrument 4400 is inside the restricted operating condition (e.g., so long as the current operating condition coincides with the restricted operating condition).

With reference to method 20, FIG. 17 depicts the insertion and/or withdrawal of the designated portion 4450 of the medical instrument 4400 through a restricted operating condition corresponding to a portion (e.g., region RC₁) of the operating range R_{O} that extends distally from the defined reference location RL on the cannula 4600 of the surgical system 1000. This region may, for example, extend distally from the defined reference location RL for 25 mm or less. As such, when the designated portion 4450) of the instrument 4400 is at IP₁, the current operating condition of the instrument 4400 is in the second condition inside the restricted operating condition. In the embodiment depicted, the force feedback coefficient is set to zero, and the haptic feedback is disabled. As the designated portion 4450 transitions from IP₁ to IP₂, the value of the force feedback coefficient may be increased in accordance with the gain function f_{(G)} resulting in an increase in the magnitude of the restricted haptic feedback HF_{R} provided to the input device 1116. Further distal movement of the instrument 4400 may result in additional increases in the force feedback coefficient in accordance with the gain function f_{(G)} until a force feedback coefficient of one is achieved. As depicted at position IP₃, the current operating condition may be outside the restricted operating condition of the instrument 4400. Therefore, the force feedback coefficient may be 1 and the nominal haptic feedback HF_{N} may be provided to the input device 1116.

With reference to method 20, FIG. 18 depicts a proximal or distal translation of the designated portion 4450 of the medical instrument 4400 through a restricted operating condition corresponding to a portion (e.g., region RC₂) of the operating range R_{O}. As depicted in FIG. 18, a proximal translation of the designated portion 4450 of the instrument 4400 may proceed from the first condition at position IP₆ until the current operating condition changes from being outside the restricted operating condition to being inside the restricted operating condition of the instrument 1400. Following the change, the force feedback coefficient may be reduced from one in accordance with the second gain function f_{(G2)}, passing through position IP₅. Further proximal movement of the designated portion 4450 of the instrument 4400 may result in a reduction of the force feedback coefficient to zero. Upon completing the transit of the restricted operating condition (e.g., region RC₂) the force feedback coefficient may be increased in accordance with the first gain function f_{(G1)}, passing through position IP₄. Continued proximal movement of the designated portion 4450 of the instrument 4400 may result in the current operating condition again being outside the restricted operating condition, thereby facilitating the delivery of the nominal haptic feedback HF_{N}.

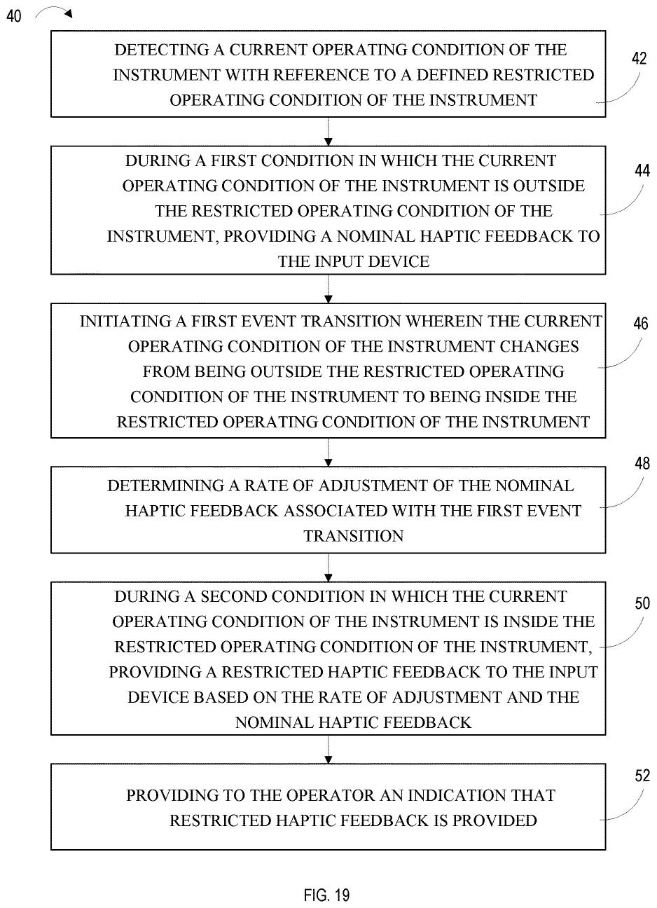

Although the method 20 is shown and described above as determining a force feedback coefficient based on the position the device relative to a restricted operating condition (i.e., the distance D described above), in other embodiments, a method can include providing a restricted haptic feedback based on any other suitable parameters, such as the time within which a restricted feedback condition has been detected, the velocity of a portion of the medical instrument, the acceleration of a portion of the medical instrument, the energy generated by a cautery instrument, or other suitable parameters. Thus, in some embodiments a method can include applying a rate of adjustment of the nominal haptic feedback. For example, FIG. 19 is a flow chart of a method 40 of control for a surgical system according to an embodiment. The method 40 may, in an embodiment, be performed via the system 1000 as described with reference to FIGS. 4-10 and FIG. 22. However, it should be appreciated that in various embodiments, aspects of the method 40 may be accomplished via additional embodiments of the system 1000 or components thereof, such as instrument 2400, instrument 3400, and/or instrument 4400) as described herein. As such, the method 40 may be implemented, at least in part, via the controller 1800 of the system 1000 described in FIGS. 4-10 and FIG. 22. Thus, the method 40 is described below with reference to medical instrument 4400 and the controller 1800 of the system 1000 depicted in FIGS. 4-10 and FIG. 22, but it should be understood that the method 40 can be employed using any of the medical devices/instruments and controllers described herein. For example, as depicted at step 42, controller 1800 (e.g., via the haptic feedback module 1820) may detect a current operating condition of the medical instrument 4400 (e.g., instruments 1400, 2400 and/or 3400). The current operating condition of the instrument 4400 may be determined with reference to a defined restricted operating condition of the instrument 4400.

The restricted operating condition may be associated with an activation of an energized medical instrument of the surgical system 1000 (e.g., via the device control module 1814 (FIG. 22)). For example, in an embodiment, the activation of the energized medical instrument may correspond to the activation of a portion of the instrument 4400 such as end effector 4460 configured to cauterize a region of the surgical site. In additional embodiments, the activation of the energized medical instrument may correspond to the activation of a sensing implement (e.g., an ultrasonic transducer), a cutting/ablating implement (e.g., an ultrasonic and/or heat-based cutting implement), and/or other similar implements activated, at least in part, via an electrical current delivered to a portion of the instrument 4400. It should be appreciated that the activation of an energized medical instrument may, under certain conditions, affect a signal produced by at least one sensor (e.g., strain sensor(s) 2830) and may therefore affect a determination of the forces acting on or through the medical instrument 4400. This, in turn, may limit the ability of the controller 1800 to provide accurate haptic feedback to the surgeon S. Accordingly, it may be desirable to mitigate the impact of the activation of the energized medical instrument on the haptic feedback.

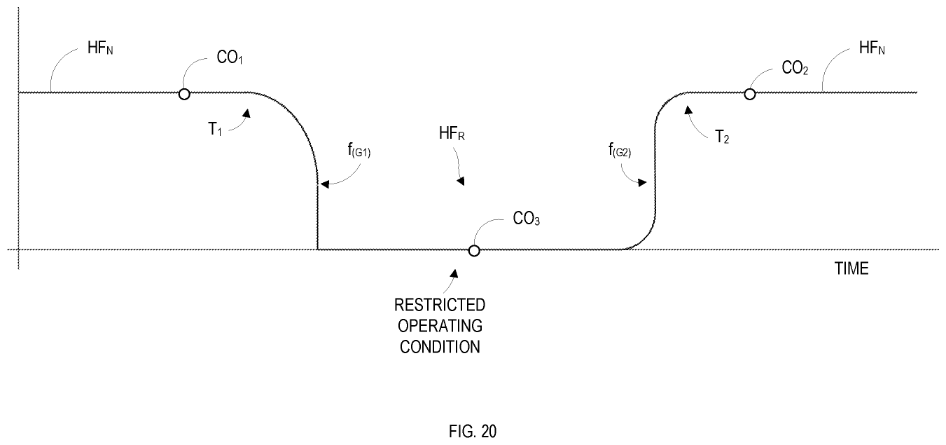

As further illustrated at step 44 in FIG. 19, in a first condition, the current operating condition of the instrument 4400 may be outside (e.g., positions CO₁ and CO₂ (FIG. 20)) the restricted operating condition of the instrument 4400. Further at step 44, when the current operating condition is outside the restricted operating condition, the controller 1800 (e.g., via the haptic feedback module 1820 (FIG. 22)) may provide the nominal haptic feedback HF_{N} to the input device 1116. The nominal haptic feedback HF_{N} may correspond to a designed/intended haptic feedback characteristic and/or magnitude delivered to the surgeon S when a restriction on the operating condition of the instrument 4400 is absent.

As depicted at step 46, a first event transition T₁ may be initiated. With the first event transition T₁, the current operating condition of the instrument 1400 may change from being outside (e.g., positions CO₁ and CO₂ (FIG. 20)) the restricted operating condition of the instrument 4400 to being inside (e.g., position CO₃ (FIG. 20)). The first event transition T₁ may be initiated, for example, when the controller 1800 receives a command signal from the operator/surgeon S of the surgical system 1000 (e.g., via the control input module 1808 (FIG. 22)). The command signal may initiate an operation that utilizes the energized medical instrument, such as a cauterization at the surgical site.

In response to the first event transition T₁, the controller 1800 may, at step 48, determine a rate of adjustment of the nominal haptic feedback HF_{N} associated with the first event transition T₁ (e.g., via the haptic feedback module 1820). The rate of adjustment may mitigate an impact of the first event transition T₁. As such, the rate of adjustment may correspond to a rate of change of a force feedback coefficient. The force feedback coefficient may, as otherwise described herein, be utilized by the controller 1800 to modify the nominal haptic feedback HF_{N} in association with the first event transition T₁. The rate of adjustment may, for example, reduce the haptic feedback delivered to the input device 1116 from a nominal haptic feedback level to a restricted haptic feedback level over a relatively short time interval so as to mitigate an impact of the restricted operating condition.

In an embodiment, the rate of adjustment, and thus the force feedback coefficient, may be defined by the controller 1800 (e.g., via the haptic feedback module 1820) based on a gain function f_{(G)} and/or the saturation function. The gain function f_{(G)} and/or the saturation function may define a curve (e.g., a feedback-transition curve) descriptive of a rate of change of the force feedback coefficient relative to a variable, such as time/duration. In an embodiment, such as depicted in FIG. 20, the gain function f_{(G)} may describe a sigmoid curve (e.g., gain function f_{(G2)}).

As described for previous embodiment, in some embodiments employing the gain function f_{(G)} to modify the nominal haptic feedback HF_{N}, the controller 1800 will multiply the sensed force (e.g., as indicated by the output of the force sensor unit 2800 or the force sensor unit 3800) by a factor that is a function of a variable, such as the distance D. However, in some embodiments a saturation function can be employed to modify the nominal haptic feedback HF_{N}, by the multiplication of a maximal haptic feedback magnitude by a factor that is a function of a variable, such as distance D and the magnitude of the sensed force ((e.g., as indicated by the output of the force sensor unit 2800 or the force sensor unit 3800). In other words, the saturation function may be employed to reduce a maximal force that may be delivered by the haptic feedback system to the operator S to a fraction of a nominal maximal haptic feedback magnitude.

By way of additional illustration, in an embodiment, the gain function f_{(G)} may describe a linear ramp. However, in an embodiment, such as further depicted in FIG. 20, the gain function f_{(G)} (described above with reference to FIGS. 17 and 18) may describe an exponential curve (e.g., gain function f_{(G1)}. In further embodiments, the gain function f_{(G)} and/or the saturation function may define other suitable curves. It should thus be appreciated that the nominal haptic feedback HF_{N} may be modified by the force feedback coefficient in accordance with the gain function f_{(G)} so that a portion may be provided to the surgeon S when the instrument 4400 encounters the corresponding variable (e.g., is at a corresponding time interval).

To define the rate of adjustment based on the gain function f_{(G)} and/or the saturation function, the controller 1800 may determine the gain function f_{(G)} and/or the saturation function based on an operating condition of the instrument 4400 or impacting the instrument 4400. In an embodiment, the operating condition may correspond to a power level of the energized medical instrument. For example, a cautery, cutting, and/or ablating operation may require relatively high power levels. As such, a relatively high rate of change of the force feedback coefficient may be desirable in order to affect a relatively rapid change in the haptic feedback so as to mitigate the impacts of the relatively high-power operation. However, when the energized medical instrument is employed to perform a relatively low-power operation, then a relatively low rate of change of the force feedback coefficient may be desirable in order to affect a more gradual change in the haptic feedback then would be observed at a higher rate of change of the force feedback coefficient. In additional embodiments, the gain function f_{(G)} and/or the saturation function may be defined by an acceleration of a portion of the instrument 4400 and/or a direction of a change in force. The gain function f_{(G)} and/or the saturation function may be tailored to achieve a change in the haptic feedback at a rate that is most beneficial for the operating condition of the instrument 4400.

As depicted in FIG. 20, in an embodiment, different gain/saturation functions f_{(G)} may be utilized purposes, such as transitioning to or transitioning from the restricted operating condition. For example, when transitioning between current operating conditions at positions CO₁ and CO₃, the rate of adjustment may be defined by a first gain function f_{(G1)} (e.g., an exponential curve). However, when transitioning between current operating conditions at positions CO₃ and CO₂, the rate of adjustment may be defined by a second gain function f_{(G2)} (e.g., a sigmoid curve).

As shown in FIG. 19, at step 50, a second condition may exist when the current operating condition (e.g., position CO₃) of the instrument 1400 is inside the restricted operating condition of the instrument 4400. Accordingly, the controller 1800 (e.g., via the haptic feedback module 1820) may, at step 50, provide a restricted haptic feedback HF_{R} to the input device 1116. In other words, whenever the current operating condition of the instrument 4400 is inside the restricted operating condition, the nominal haptic feedback HF_{N} may be reduced and/or disabled. The restricted haptic feedback HF_{R} may, for example, be based on the rate of adjustment and the nominal haptic feedback HF_{N}.

The restricted haptic feedback HF_{R} may, for example, be determined based on the nominal haptic feedback HF_{N} and the rate of adjustment. It should therefore be appreciated that the force feedback coefficient may facilitate the delivery of level of haptic feedback to the surgeon S that is a percentage of the nominal haptic feedback HF_{N} that is less than 100%. This may include delivering zero percent of the nominal haptic feedback (e.g., disabling, at least temporarily, the haptic feedback system).

As depicted at step 52, in conjunction with providing the restricted haptic feedback HF_{R}, the controller 1800 may provide the operator/surgeon S an indication that restricted haptic feedback is being provided to the input device 1116 (e.g., via the indicator module 1812 (FIG. 22)). In other words, on the first event transition T₁ may, the operator/surgeon S may receive an indication that the haptic feedback provided to the input device 1116 is deviating from the nominal haptic feedback HF_{N}.

In an embodiment, the delivery of the restricted haptic feedback HF_{R} may be accomplished automatically (e.g., without operator S input) coincident with notification to the operator/surgeon S. For example, in an embodiment, the controller 1800 may be configured to automatically provide the restricted haptic feedback HF_{R} to the input device 1116 in accordance with a feedback-restriction interval. The feedback-restriction interval may be based on a nominal duration of the activation of the energized medical device. For example, the activation of the energized medical device may initiate a cautery operation having a preset duration.

In an embodiment, such as depicted in FIG. 20, the rate of adjustment of step 48 may be a first rate of adjustment. In such an embodiment, a second event transition T₂ may be initiated. With the second event transition T₂, the current operating condition of the instrument 1400 may change from the second condition (e.g., the current operating condition being inside the restricted operating condition, such as during the activation of the energized medical instrument) to the first condition (e.g., the current operating condition being outside the restricted operating condition). In other words, wherein the first event transition T₁ may indicate a departure from the nominal operating condition corresponding to the activation of the energized medical instrument, the second event transition T₂ may indicate the completion of the energized operation and a return to a nominal operating condition from the restricted operating condition.

In association with the second event transition T₂, the controller 1800 (e.g., the haptic feedback module 1820) may determine a corresponding rate of adjustment. In accordance with the rate of adjustment, the controller 1800 may transition the restricted haptic feedback HF_{R} to the nominal haptic feedback HF_{N}. In an embodiment wherein the rate of adjustment corresponding to the first event transition T₁ is based on the first gain function f_{(G1)}, the rate of adjustment corresponding to the second event transition may be based on a second gain function f_{(G2)}. It should be appreciated that employing different rates of adjustment corresponding to different gain functions may facilitate the tailoring of the haptic feedback delivered to the input device 1116 to different operations of the surgical system 1000.

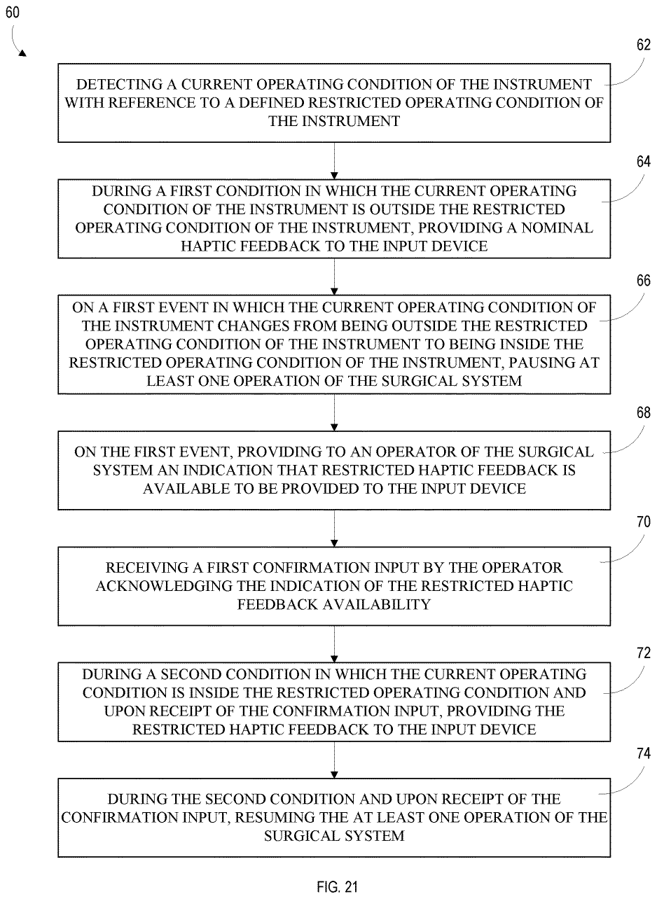

FIG. 21 is a flow chart of a method 60 of control for a surgical system according to an embodiment. The method 60 may, in an embodiment, be performed via the system 1000 as described with reference to FIGS. 4-10 and FIG. 22. However, it should be appreciated that in various embodiments, aspects of the method 60 may be accomplished via additional embodiments of the system 1000 or components thereof, such as instrument 2400, instrument 3400, and/or instrument 4400 as described herein. As such, the method 60 may be implemented, at least in part, via the controller 1800 of the system 1000 described in FIGS. 4-10 and FIG. 22. Thus, the method 40 is described below with reference to medical instrument 4400 and the controller 1800 of the system 1000 depicted in FIGS. 4-10 and FIG. 22, but it should be understood that the method 40 can be employed using any of the medical devices/instruments and controllers described herein. For example, as depicted at 62, controller 1800 may detect a current operating condition of the medical instrument 4400 (e.g., instrument 2400 and/or instrument 3400). The current operating condition of the instrument 1400 may be determined with reference to a defined restricted operating condition of the instrument 1400.

As similarly described above in reference to embodiments depicted in FIGS. 16-17, in an embodiment, the restricted operating condition may correspond to a positioning of a designated portion 4450 of the instrument 4400. Specifically, the restricted operating condition may correspond to a restricted operating region. The restricted operating region may correspond to a defined distance between the designated portion 4450 of the instrument 4400 and the defined reference location RL on the cannula of the surgical system 1000. However, in an additional embodiment, and as also described above, the restricted operating condition may be associated with an activation of the energized medical instrument of the surgical system 1000.

In a further embodiment, the restricted operating condition may correspond to a fault condition. As such, in an embodiment, the controller 1800 may be configured to receive a communication signal associated with the fault condition. The fault condition may be a sensor fault, a communication fault, and/or a haptic system fault. It should be appreciated that generating haptic feedback when at least one of the above faults is present may be undesirable. Accordingly, mitigating the impact of the fault condition on the haptic feedback provided to the operator S may be desirable.

As illustrated at step 64 in FIG. 21, in a first condition, the current operating condition of the instrument 4400 may be outside the restricted operating condition of the instrument 4400. Further, at step 64, when the current operating condition is outside the restricted operating condition, the controller 1800 (e.g., via the haptic feedback module 1820) may provide the nominal haptic feedback HF_{N} to the input device 1116. The nominal haptic feedback HF_{N} may correspond to a designed/intended haptic feedback characteristic and/or magnitude delivered to the surgeon S when a restriction on the operating condition of the instrument 4400 is absent.

As shown at step 66, on the occurrence of a first event, the current operating condition of the instrument 4400 may change/transition from being outside the restricted operating condition to being inside the restricted operating condition of the instrument 4400. Upon detecting such a change/transition, the controller 1800 may, at step 66, pause at least one operation of the surgical system 1000. For example, on the occurrence of the first event, the controller 1800 may halt or freeze all movements and/or operations of the surgical system 1000 that impact the instrument 4400. It should be appreciated that halting or freezing the movement/operations of the surgical system 1000 upon the transition to the restricted operating condition may preclude the operation of the surgical system in the presence of limited/degraded haptic feedback. Therefore, the pausing of the at least one operation may facilitate the effective utilization of the surgical system 1000.

Concurrent with pausing the at least one operation of the surgical system 1000, the controller 1800 may, at step 68, provide the operator/surgeon S an indication that restricted haptic feedback HF_{R} is available to be provided to the input device 1116. In other words, on the first event, the operator/surgeon S may receive an indication that the at least one operation of the surgical system 1000 is paused and that the haptic feedback available to be provided to the input device 1116 may deviate from the nominal haptic feedback HF_{N}.

The indication that the restricted haptic feedback HF_{R} is available may be a visual indication, a haptic indication, and/or an audible indication. For example, the controller 1800 may, in an embodiment, be configured to generate a graphical indication of a deviation of the restricted haptic feedback HF_{R} from the nominal haptic feedback HF_{N}. The controller 1800 may maintain (e.g., via the display system 1110) the graphical indication within a field-of-view 1142 of the operator/surgeon S when the instrument 4400 is inside the restricted operating condition (e.g., so long as the current operating condition coincides with the restricted operating condition).

While maintaining the at least one operation as paused, the controller 1800 may, at step 70, be configured to receive a first confirmation input by the operator S (e.g., via the control input module 1808 (FIG. 22)). The first confirmation input may acknowledge the indication of the restricted haptic feedback HF_{R} availability. In an embodiment, the confirmation input may include an operator gesture, an engagement between the operators head and a user interface of the surgical system 1000, a touchpad input, a button activation, a pedal activation, a button, and pedal activation combination, and/or a crossing of a haptic barrier. For example, in response to the indication being displayed within the field-of-view 1142 (see FIG. 8), the surgeon S may input the confirmation via the pedal assembly 1136 and/or foot-activated switch assembly 1138, both shown in FIG. 6.

As depicted at 72, the controller 1800 (e.g., the haptic feedback module 1820) may be configured to provide the restricted haptic feedback HF_{R} to the input device 1116. The restricted haptic feedback HF_{R} may be provided during a second condition in which the current operating condition is inside the restricted operating condition and upon receipt of the confirmation input. In an embodiment, providing the restricted haptic feedback HF_{R} may include limiting the restricted haptic feedback HF_{R} to a percentage of a nominal haptic feedback level. In embodiment, the percentage may be less than 100%. In an additional embodiment, the percentage may be 0%.

Additionally, during the second condition and upon receipt of the confirmation input, the controller 1800 may be configured to, at step 74, resume the at least one operation of the surgical system 1000. In other words, the paused operation of the surgical system 1000 may remain in a paused state until the confirmation input is received from the surgeon S. It should be appreciated that maintaining the surgical system 1000 in a paused state may preclude an operation of the instrument 4400 when degraded haptic feedback may be provided to the operator/surgeon S. Therefore, the pausing of the operation may facilitate the effective transition between nominal operating conditions and restricted operating conditions.

In an embodiment, limiting the restricted haptic feedback HF_{R} to a percentage of the nominal haptic feedback level may include dynamically reducing the percentage of the nominal haptic feedback level based on a gain function and/or a saturation function. As previously described, the gain function may define a curve (e.g., a feedback-transition curve) descriptive of a rate of change of the force feedback coefficient relative to a variable, such as distance and/or time. As also described above, the controller 1800 may determine the gain function f_{(G)} and/or the saturation function based on an operating condition of the instrument 4400 or impacting the instrument 4400.

As described above for previous embodiments, in some embodiments employing the gain function f_{(G)} to modify the nominal haptic feedback HF_{N}, the controller 1800 will multiply the sensed force (e.g., as indicated by the output of the force sensor unit 2800 or the force sensor unit 3800) by a factor that is a function of a variable, such as the distance D. However, in some embodiments employing the saturation function to modify the nominal haptic feedback HF_{N}, the controller 1800 will multiply a maximal haptic feedback magnitude by a factor that is a function of a variable, such as distance D and the magnitude of the sensed force ((e.g., as indicated by the output of the force sensor unit 2800 or the force sensor unit 3800). In other words, the saturation function may be employed to reduce a maximal force that may be delivered by the haptic feedback system to the operator S to a fraction of a nominal maximal haptic feedback magnitude.

In addition to transitioning from a nominal operating condition to a restricted operating condition, in an embodiment, the instrument 4400 may transition from the restricted operating condition to a nominal operating condition. Accordingly, on a second event in which the current operating condition of the instrument 1400 changes from being inside the restricted operating condition of the instrument 4400 to being outside the restricted operating condition instrument, the controller 1800 may pause the at least one operation of the surgical system 1000. The at least one operation of the surgical system 1000 may be the same operation(s) that was paused in conjunction with the first event. In other words, prior to returning the feedback level to the nominal feedback level, the operation(s) may be paused in order to ensure an effective transition between the operating conditions.

Also on the second event, the controller 1800 may provide the operator S of the surgical system 1000 with an indication that the nominal haptic feedback HF_{N} is available to be provided to the input device 1116. Accordingly, the controller 1800 may receive a second confirmation input by the operator S acknowledging the indication of the nominal haptic feedback availability. Upon receipt of the second confirmation and during the first condition, the controller 1800 (e.g., haptic feedback module 1820) may provide the nominal haptic feedback HF_{N} to the input device 1116 and resume the at least one operation of the surgical system 1000. In an embodiment, providing the nominal haptic feedback may include increasing, via the controller 1800 (e.g., haptic feedback module 1820) a haptic feedback level from a restricted haptic feedback level to a nominal haptic feedback level according to a gain function as previously described herein.

As shown particularly in FIG. 22, a schematic diagram of one embodiment of suitable components that may be included within the controller 1800 is illustrated. In some embodiments, the controller 1800 is positioned within a component of the surgical system 1000, such as the user control unit 1100 and/or the optional auxiliary equipment unit 1150. However, the controller 1800 may also include distributed computing systems wherein at least one aspect of the controller 1800 is at a location which differs from the remaining components of the surgical system 1000 for example, at least a portion of the controller 1800 may be an online controller.

As depicted, the controller 1800 includes one or more processor(s) 1802 and associated memory device(s) 1804 configured to perform a variety of computer implemented functions (e.g., performing the methods, steps, calculations and the like and storing relevant data as disclosed herein). Additionally, in some embodiments, the controller 1800 includes a communication module 1806 to facilitate communications between the controller 1800 and the various components of the surgical system 1000.

As used herein, the term “processor” refers not only to integrated circuits referred to in the art as being included in a computer, but also refers to a controller, a microcontroller, a microcomputer, a programmable logic controller (PLC), an application specific integrated circuit, and other programmable circuits. Additionally, the memory device(s) 1804 may generally comprise memory element(s) including, but not limited to, computer readable medium (e.g., random access memory (RAM)), computer readable nonvolatile medium (e.g., a flash memory), a floppy disk, a compact disc read only memory (CD ROM), a magneto optical disk (MOD), a digital versatile disc (DVD) and/or other suitable memory elements. Such memory device(s) 1804 may generally be configured to store suitable computer readable instructions that, when implemented by the processor(s) 1802, configure the controller 1800 to perform various functions.

In some embodiments, the controller 1800 includes a haptic feedback module 1820. The haptic feedback module 1820 may be configured to deliver a haptic feedback to the operator S based on inputs received from a force sensor unit of the instrument 1400 (e.g., the force sensor unit 3800, including the strain sensors 3830 (FIG. 13). In some embodiments, haptic feedback module 1820 may be an independent module of the controller 1800. However, in some embodiments the haptic feedback module 1820 may be included within the memory device(s) 1804.

The communication module 1806 may include a control input module 1808 configured to receive control inputs from the operator/surgeon S, such as via the input device 1116 of the user control unit 1100. The communication module may also include an indicator module 1812 configured to generate various indications in order to alert the operator S.

The communication module 1806 may also include a sensor interface 1810 (e.g., one or more analog to digital converters) to permit signals transmitted from one or more sensors (e.g., strain sensors 2830 of the force sensor unit 2800 (FIG. 11) to be converted into signals that can be understood and processed by the processors 1802. The sensors may be communicatively coupled to the communication module 1806 using any suitable means. For example the sensors may be coupled to the communication module 1806 via a wired connection and/or via a wireless connection, such as by using any suitable wireless communications protocol known in the art. Additionally, in some embodiments, the communication module 1806 includes a device control module 1814 configured to modify an operating state of the instrument 1400 (and/or any of the instruments described herein (e.g., 2400, 3400, 4400). Accordingly, the communication module is communicatively coupled to the manipulator 1200 and/or the instrument 1400. For example, the communications module 1806 may communicate to the manipulator 1200 and/or the instrument 1400 an excitation voltage for the strain sensor(s), a handshake and/or excitation voltage for a positional sensor (e.g., for detecting the position of the designated portion relative to the cannula), cautery controls, positional setpoints, and/or an end effector operational setpoint (e.g., gripping, cutting, and/or other similar operation performed by the end effector).

While various embodiments have been described above, it should be understood that they have been presented by way of example only, and not limitation. Where methods and/or schematics described above indicate certain events and/or flow patterns occurring in certain order, the ordering of certain events and/or operations may be modified. While the embodiments have been particularly shown and described, it will be understood that various changes in form and details may be made.

For example, any of the instruments described herein (and the components therein) are optionally parts of a surgical assembly that performs minimally invasive surgical procedures, and which can include a manipulator unit, a series of kinematic linkages, a series of cannulas, or the like. Thus, any of the instruments described herein can be used in any suitable surgical system, such as the MIRS system 1000 shown and described above. Moreover, any of the instruments shown and described herein can be used to manipulate target tissue during a surgical procedure. Such target tissue can be cancer cells, tumor cells, lesions, vascular occlusions, thrombosis, calculi, uterine fibroids, bone metastases, adenomyosis, or any other bodily tissue. The presented examples of target tissue are not an exhaustive list. Moreover, a target structure can also include an artificial substance (or non-tissue) within or associated with a body, such as for example, a stent, a portion of an artificial tube, a fastener within the body or the like.

For example, any of the components of a surgical instrument as described herein can be constructed from any material, such as medical grade stainless steel, nickel alloys, titanium alloys or the like. Further, any of the links, tool members, beams, shafts, cables, or other components described herein can be constructed from multiple pieces that are later joined together. For example, in some embodiments, a link can be constructed by joining together separately constructed components. In other embodiments however, any of the links, tool members, beams, shafts, cables, or components described herein can be monolithically constructed.

Although various embodiments have been described as having particular features and/or combinations of components, other embodiments are possible having a combination of any features and/or components from any of embodiments as discussed above. Aspects have been described in the general context of medical devices, and more specifically surgical instruments, but inventive aspects are not necessarily limited to use in medical devices.

1. A method of control for a computer-assisted system, the computer-assisted system including a controller, an input device, and an instrument operably coupled to be controlled by the input device via the controller, the method comprising:
detecting, via the controller, a current operating condition of the instrument with reference to a defined restricted operating condition of the instrument;
determining, via the controller, a force feedback coefficient based on the current operating condition of the instrument;
determining, via the controller, a restricted haptic feedback based on a design haptic feedback and on the force feedback coefficient;
during a first condition in which the current operating condition of the instrument is outside the restricted operating condition of the instrument, providing, via the controller, the design haptic feedback to the input device; and
on a first event in which the current operating condition of the instrument changes from being outside the restricted operating condition of the instrument to being inside the restricted operating condition of the instrument, providing, via the controller, to an operator of the computer-assisted system an indication that restricted haptic feedback is provided to, or is available to be provided to, the input device.

2. The method of claim 1, wherein:
during a second condition in which the current operating condition of the instrument is inside the restricted operating condition of the instrument, the method comprises providing, via the controller, the restricted haptic feedback to the input device.

3. The method of claim 1, wherein:
during a second condition in which the current operating condition is inside the restricted operating condition, and on a second event in which an input by the operator acknowledging the indication of the restricted feedback is received at the controller, the method comprises providing, via the controller, the restricted haptic feedback to the input device.

4. The method of claim 3, wherein:
providing the restricted haptic feedback to the input device includes providing a haptic feedback level that is less than a design haptic feedback level.

5. The method of claim 1, wherein:
the restricted operating condition is a portion of an operating range of the instrument in which a determination by the controller of a force acting on the instrument deviates from the actual force acting on the instrument.

6. The method of claim 1, wherein:
detecting the current operating condition of the instrument includes determining, via the controller, a distance between a portion of the instrument and a defined reference location on a cannula of the computer-assisted system.

7.-9. (canceled)

10. The method of claim 1 wherein:
providing the design haptic feedback includes generating the design haptic feedback based on a strain sensor signal from the instrument received at the controller.

11. A method of control for a computer-assisted system, the computer-assisted system including a controller, an input device, and an instrument operably coupled to be controlled by the input device via the controller, the method comprising:
detecting, via the controller, a current operating condition of the instrument with reference to a defined restricted operating condition of the instrument;
during a first condition in which the current operating condition of the instrument is outside the restricted operating condition of the instrument, providing, via the controller, a design haptic feedback to the input device;
initiating a first event transition wherein the current operating condition of the instrument changes from being outside the restricted operating condition of the instrument to being inside the restricted operating condition of the instrument;
determining, via the controller, a rate of adjustment of the design haptic feedback associated with the first event transition;
during a second condition in which the current operating condition of the instrument is inside the restricted operating condition of the instrument, providing, via the controller, a restricted haptic feedback to the input device based on the rate of adjustment and the design haptic feedback; and
providing to an operator of the computer-assisted system, via the controller, an indication that restricted haptic feedback is provided.

12. The method of claim 11, wherein:
the restricted operating condition is associated with an activation of an energized instrument of the computer-assisted system.

13. The method of claim 11, wherein:
initiating the first event transition includes receiving, via the controller, a command signal from the operator of the computer-assisted system initiating an operation that utilizes the energized instrument.

14. The method of claim 12, wherein:
providing the restricted haptic feedback includes establishing the haptic feedback in accordance with a feedback-restriction interval, the feedback-restriction interval being based on a design duration of the activation of the energized instrument.

15.-16. (canceled)

17. The method of claim 11, wherein:
establishing the haptic feedback delivered to the operator includes establishing a level of the haptic feedback delivered to the operator at a value less than a design haptic feedback level.

18. The method of claim 11, wherein:
the rate of adjustment is a first rate of adjustment; and
the method further comprises:
initiating a second event transition wherein the current operating condition of the instrument changes from the second condition to the first condition,
determining, via the controller, a rate of adjustment of the restricted haptic feedback associated with the second event transition, and
transitioning, via the controller, the restricted haptic feedback to the design haptic feedback in accordance with the rate of adjustment.

19.-20. (canceled)

21. A method of control for a computer-assisted system, the computer-assisted system including a controller, an input device, and an instrument operably coupled to be controlled by the input device via the controller, the method comprising:
detecting, via the controller, a current operating condition of the instrument with reference to a defined restricted operating condition of the instrument;
during a first condition in which the current operating condition of the instrument is outside the restricted operating condition of the instrument, providing, via the controller, a design haptic feedback to the input device;
on a first event in which the current operating condition of the instrument changes from being outside the restricted operating condition of the instrument to being inside the restricted operating condition of the instrument, pausing, via the controller, an operation of the computer-assisted system;
on the first event, providing to an operator of the computer-assisted system, via the controller, an indication that restricted haptic feedback is available to be provided to the input device;
receiving, via the controller, a first confirmation input by the operator acknowledging the indication of the restricted haptic feedback availability;
during a second condition in which the current operating condition is inside the restricted operating condition and upon receipt of the confirmation input, providing, via the controller, the restricted haptic feedback to the input device; and
during the second condition and upon receipt of the confirmation input, resuming, via the controller, the operation of the computer-assisted system.

22. The method of claim 21, further comprising:
on a second event in which the current operating condition of the instrument changes from being inside the restricted operating condition of the instrument to being outside the restricted operating condition of the instrument, pausing, via the controller, the operation of the computer-assisted system;
on the second event, providing to the operator of the computer-assisted system, via the controller, an indication that the design haptic feedback is available to be provided to the input device;
receiving, via the controller, a second confirmation input by the operator acknowledging the indication of the design haptic feedback availability;
during the first condition and upon receipt of the second confirmation input, providing, via the controller, the design haptic feedback to the input device; and
during the first condition and upon receipt of the second confirmation input, resuming, via the controller, the operation of the computer-assisted system.

23. The method of claim 22, wherein:
on the second event, providing the design haptic feedback includes increasing, via the controller, a haptic feedback level from a restricted haptic feedback level to a design haptic feedback level according to a gain function.

24. The method of claim 21, wherein:
the current operating condition of the instrument is inside the restricted operating condition when a position of a designated portion of the instrument is within a restricted operating region; and
the restricted operating region corresponds to a defined distance between the designated portion of the instrument and a defined reference location on a cannula of the computer-assisted system.

25. (canceled)

26. The method of claim 21, wherein:
the restricted operating condition corresponds to a fault condition;
the method further comprises receiving, via the controller, a communication signal associated with the fault condition; and
the fault condition is at least one of a sensor fault, a communications fault, or a haptic system fault.

27. (canceled)

28. The method of claim 21, wherein:
providing the restricted haptic feedback includes limiting the restricted haptic feedback to less than 100 percent of a design haptic feedback level.

29. The method of claim 28, wherein:
limiting the restricted haptic feedback to a percentage of the design haptic feedback level includes dynamically reducing the percentage of the design haptic feedback level based on a gain function.

30.-56. (canceled)
